(19) United States
(12) Patent Application Publication
(10) Pub. No.: US 2023/0165882 A1
(43) Pub. Date: Jun. 1, 2023

(54) COMPOUNDS AND METHODS FOR REDUCTION OF CANCER CELL BURDEN AND PROTECTION OF NORMAL HEMATOPOIESIS

(71) Applicant: MAGNANI, John L., Gaithersburg, MD (US)

(72) Inventors: John L. MAGNANI, Gaithersburg, MD (US); William E. FOGLER, Lutherville-Timonium, MD (US)

(73) Assignee: GLYCOMIMETICS, INC., Rockville, MD (US)

(21) Appl. No.: 17/998,942

(22) PCT Filed: May 28, 2021
(86) PCT No.: PCT/US2021/034773

§ 371 Date: Nov. 16, 2022

Related U.S. Application Data
(60) Provisional application No. 63/032,675, filed on May 31, 2020.
(60) Provisional application No. 63/060,595, filed on Aug. 3, 2020.
(60) Provisional application No. 63/063,892, filed on Aug. 10, 2020.
(60) Provisional application No. 63/198,864, filed on Nov. 18, 2020.

(51) Int. Cl. A61K 31/7034; A61P 35/02; A61K 31/7052; A61K 31/44

(52) U.S. Cl. CPC A61K 31/7034; A61P 35/02; A61K 31/7052; A61K 31/44

(57) ABSTRACT

Methods for treating cancer (such as, e.g., acute myelogenous leukemia), enhancing maintenance of normal hematopoiesis in bone marrow, and/or mobilizing leukemia blasts in a subject in need thereof comprising administering to the subject at least one FLT3 inhibitor and at least one inhibitor chosen from E-selectin inhibitors, CXCR4 inhibitors, and heterobifunctional inhibitors of E-selectin and CXCR4.

This application claims the benefit of priority of U.S. Provisional Application No. 63/032,675, filed May 31, 2020, U.S. Provisional Application No. 63/060,595, filed Aug. 3, 2020, U.S. Provisional Application No. 63/063,892, filed Aug. 10, 2020, and U.S. Provisional Application No. 63/198,864, filed Nov. 18, 2020, the contents of each of which are herein incorporated by reference in their entirety.

Disclosed herein are methods for treating a cancer (such as, e.g., acute myelogenous leukemia), enhancing maintenance of normal hematopoiesis in bone marrow, and/or mobilizing leukemia blasts in a subject in need thereof comprising administering to the subject at least one FLT3 inhibitor and at least one inhibitor chosen from E-selectin inhibitors, CXCR4 inhibitors, and heterobifunctional inhibitors of E-selectin and CXCR4.

Selectins are a class of cell adhesion molecules that have well-characterized roles in leukocyte homing. These cell adhesion molecules are type 1 membrane proteins and are composed of an amino terminal lectin domain, an epidermal growth factor (EGF)-like domain, a variable number of complement receptor related repeats, a hydrophobic domain spanning region and a cytoplasmic domain. Binding interactions appear to be mediated by contact of the lectin domain of the selectins and various carbohydrate ligands.

There are three known selectins: E-selectin; P-selectin; and L-selectin. E-selectin (endothelial selectin) is a transmembrane adhesion protein expressed on the surface of activated endothelial cells, which line the interior wall of capillaries. E-selectin binds to the carbohydrate sialyl-Lewis^{x} (sLe^{x}), which is presented as a glycoprotein or glycolipid on the surface of certain leukocytes (monocytes and neutrophils) and helps these cells adhere to capillary walls in areas where surrounding tissue is infected or damaged. In addition, E-selectin binds to sialyl-Lewis^{x} (sLe^{a}), which is expressed on many tumor cells. P-selectin is expressed on inflamed endothelium and platelets, and also recognizes sLe^{x} and sLe^{a}, but additionally contains a second site that interacts with sulfated tyrosine. The expression of E-selectin and P-selectin is generally increased when the tissue adjacent to a capillary is infected or damaged. L-selectin is expressed on leukocytes.

A number of cancers are treatable before the cancer has moved beyond the primary site. However, once the cancer has spread beyond the primary site, the treatment options may be limited and the survival statistics may decline dramatically. Recent investigations have suggested that cancer cells are immunostimulatory and interact with selectins to extravasate and metastasize.

Based on estimated incidence data, the most common types of cancer include prostate, breast, lung, colorectal, melanoma, bladder, non-Hodgkin's lymphoma, kidney, thyroid, leukemias, endometrial, and pancreatic cancers. The cancer with the highest expected incidence is prostate cancer. The highest mortality rate is for patients who have lung cancer. Despite enormous investments of financial and human resources, cancers such as colorectal cancer remain a leading cause of death. Illustratively, colorectal cancer is the second leading cause of cancer-related deaths in the United States among cancers that affect both men and women. Over the last several years, more than 50,000 patients with colorectal cancer have died annually.

The four most common hematological cancers are acute lymphocytic leukemia (ALL), chronic lymphocytic leukemia (CLL), chronic myelogenous leukemia (CML) and acute myelogenous leukemia (AML). Leukemias and other cancers of the blood, bone marrow, and lymphatic system affect 10 times more adults than children. However, leukemia is one of the most common childhood cancers, and 75% of childhood leukemias are ALL.

Acute myelogenous leukemia (AML) is a hematological malignancy characterized by the accumulation of abnormal immature white blood cells. AML symptoms may include fatigue, shortness of breath, easy bruising and bleeding, and increased risk of infection. It is an acute form of leukemia, which can progress rapidly and is typically fatal within weeks or months if left untreated. AML is the most common leukemia in adults. Approximately 47,000 new cases are diagnosed every year, and approximately 23,500 people die every year from leukemia. The 5-year survival rate for AML is 27.4%, with AML accounting for roughly 1.8% of cancer deaths in the United States. Acute myelogenous leukemia is also referred to as acute myeloid leukemia.

First-line treatment of AML consists primarily of chemotherapy with an anthracycline/cytarabine combination and is divided into two phases: induction and post-remission (or consolidation) therapy. The goal of induction therapy is to achieve a complete remission by reducing the number of leukemic cells to an undetectable level; the goal of consolidation therapy is to eliminate any residual undetectable disease and achieve a cure. The specific genetic mutations present within the cancer cells may guide therapy, as well as determine how long that person is likely to survive.

Despite advances in our understanding of the pathogenesis of AML, the short- and long-term outcomes for AML patients have remained unchanged over three decades (Roboz et al., 2012). The median age at diagnosis is 66 years with cure rates of less than 10% and median survival of less than 1 year (Burnett et al., 2010). Although 70-80% of patients younger than 60 years achieve complete remission, most eventually relapse, and overall survival is only 40-50% at 5 years (Fernandez et al., 2009; Mandelli et al., 2009; Ravandi et al., 2006). Relapse is thought to occur due to leukemic stem cells that escape initial induction therapy and drive reoccurrence of AML (Dean et al., 2005; Guan et al., 2003; Konopleva et al., 2002). Chemoresistance, the ability of cancer cells to evade or to cope in the presence of therapeutics, is also a key challenge for therapeutic success.

Internal tandem duplications in the Fms-like tyrosine kinase 3 (FLT3-ITD) account for 30% of adult AML cases and confer poor prognosis (Nakao et al., 1996; Kottaridis et al., 2003; Thiede et al., 2002). FLT3 inhibitors like sorafenib efficiently eliminate leukemia blast in the peripheral blood (PB), but frequently not in the bone marrow (BM) (Zhang et al., 2008). This suggests a protective effect of the BM on leukemic stem cells, which is mediated by E-selectin and CXCL12 on endothelial cells (ECs) and mesenchymal stem cells (MSCs) in the BM vascular and endosteal niches (Chien et al., 2013; Horacek et al., 2013; Peled and Tavor, 2013).

The hallmark of the AML cells containing mutations in the FLT3 gene is the constitutive kinase activation of these cancer cells. The FLT3-ITD mutation in AML patients is significantly associated with the expression of E-selectin (Kupsa et al., 2016). Specifically, the correlation of higher E-selectin expression in patients containing the FLT3-ITD mutation in their AML cells is strongly significant (P=0.0010) (Kupsa et al., 2016).

Gene expression of FUT7, an E-selectin ligand glycosylation gene, correlates to expression of the E-selectin ligand (sialyl Le^{x}) on the surface of AML cells in patients. FUT7 codes for the fucosyltransferase that adds the terminal fucose required for binding activity of the E-selectin ligand. In an analysis of a public database of AML patients, which is known as TCGA (The Cancer Genome Atlas) from NCI containing 151 paired data with Overall Survival, poor survival was only observed in FLT3-ITD AML patients that express the E-selectin ligand as determined by FUT7 expression. (See PCT International Publication No. WO 2021/011435, which is incorporated by reference herein.) Correlation of poor survival with expression of the E-selectin ligand as determined by FUT7 expression in FLT3-ITD patients is statistically significant (P=0.015), suggesting that the binding of AML cells to E-selectin drives the poor survival observed with AML patients with FLT3 mutations.

AML cells residing in BM receive a great deal of protection from the cytotoxic effects of therapeutic agents. In contrast, the circulating leukemia cells are typically more chemosensitive compared to those embedded in BM niches. The BM homing of AML cells is mediated by multiple adhesive and chemokinetic interactions including, respectively, by sialylated glycoproteins on the cancer cells binding to E-selectin on the endothelium and by CXCR4-mediated sensing of SDF-1/CXCL12 gradients. In many respects, BM homing signals are shared between leukemia and hematopoietic stem cells (HSCs).

Compounds useful for combinatorial targeting of CXCR4, E-selectin, and/or FLT3 may include Compound A:

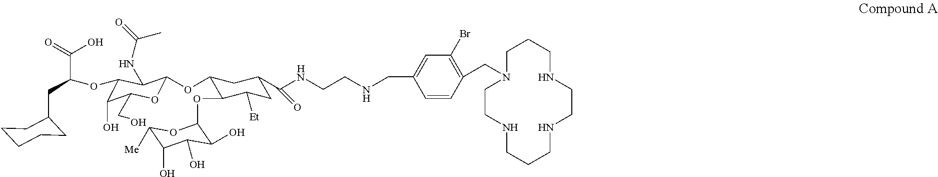 [SMILES: CCC1CC(C(=O)NCCNCc2ccc(CN3CCCNCCNCCCNCC3)c(Br)c2)CC(OC2OC(CO)C(O)C(O[C@@H](CC3CCCCC3)C(=O)O)C2NC(C)=O)C1OC1OC(C)C(O)C(O)C1O]

and/or pharmaceutically acceptable salts thereof, as well as Compound B:

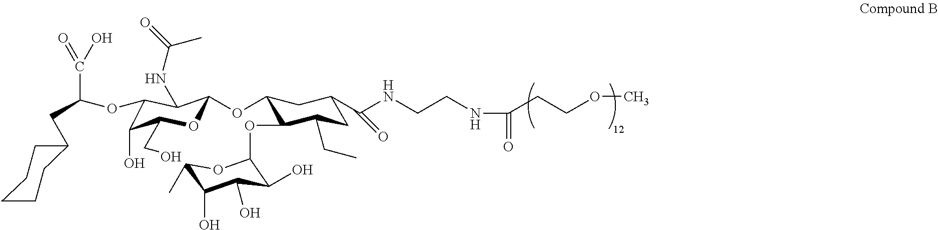

and/or pharmaceutically acceptable salts thereof.

Previous studies demonstrated that targeting E-selectin and CXCR4 with the dual E-selectin/CXCR4 antagonist Compound A markedly reduced leukemia cell adhesion to ECs and MSCs and eliminated the BM-mediated protection of leukemic cells during FLT3-targeted therapy in vitro, and effectively reduced leukemia cells in the BM in vivo (Zhang et al., 2016). Further, Compound A combined with cytarabine and daunorubicin provided a pronounced survival benefit in FLT3-mutated leukemia cell, MV4-11-bearing mice (Zhang et al., 2015).

Combinatorial targeting of CXCR4, E-selectin, and FLT3 may be effective in leukemia blast mobilization and may increase killing by abrogating BM-mediated protection. Further, this regimen may enhance maintenance of normal hematopoiesis in the BM. (FIG. 1A).

In the following description, certain specific details are set forth in order to provide a thorough understanding of various embodiments. However, one skilled in the art will understand that the disclosed embodiments may be practiced without these details. In other instances, well-known structures have not been shown or described in detail to avoid unnecessarily obscuring descriptions of the embodiments. These and other embodiments will become apparent upon reference to the following detailed description and attached drawings.

It should be understood that references herein to methods of treatment (e.g., methods of treating a cancer, such as, e.g., AML) using at least one FLT3 inhibitor and at least one inhibitor chosen from E-selectin inhibitors, CXCR4 inhibitors, and heterobifunctional inhibitors of E-selectin and CXCR4 should also be interpreted as references to:
at least one FLT3 inhibitor and at least one inhibitor chosen from E-selectin inhibitors, CXCR4 inhibitors, and heterobifunctional inhibitors of E-selectin and CXCR4 for use in methods of treating, e.g., a cancer, such as, e.g., AML; and/or
the use of at least one FLT3 inhibitor and at least one inhibitor chosen from E-selectin inhibitors, CXCR4 inhibitors, and heterobifunctional inhibitors of E-selectin and CXCR4 in the manufacture of a medicament for treating, e.g., a cancer, such as, e.g., AML.

### BRIEF DESCRIPTION OF THE DRAWINGS

FIG. 1A is a diagram illustrating a potential mechanism of combinatorial targeting of CXCR4, E-selectin, and FLT3 by Compound A and sorafenib.

FIG. 1B is a chart showing expression levels of CXCR4 and E-selectin ligands in murine leukemia cells harboring FLT3-ITD or dual FLT3-ITD plus TKD mutations exposed to either quizartinib or sorafenib for 96 hours in vitro.

FIG. 1C is a chart showing relative mRNA expression levels of CXCR4, CD162, and CD44 in murine leukemia cells harboring FLT3-ITD or dual FLT3-ITD plus TKD mutations exposed to either quizartinib or DMSO for 96 hours in vitro.

FIG. 2A is a chart showing expression levels of E-selectin ligand CD162 and CXCR4 in murine leukemia cells harboring FLT3-ITD or dual FLT3-ITD plus TKD mutations.

FIG. 2B is a chart showing expression levels of E-selectin ligand CD162 and CXCR4 in human FLT3-ITD mutated AML cells.

FIG. 3A is a chart showing the effect on leukemia cell MOLM14 adhesion to BM niche components (E-selectin, SDF-la, and collagen type I) after 20 hours of Compound B, Compound A, or plerixafor treatment.

FIG. 3B is a chart showing the effect on leukemia cell migration to NMSC/HUVEC cells after 16 hours of treatment with E-selectin inhibitor Compound B or E-selectin/CXCR4 inhibitor Compound A.

FIG. 3C is a chart showing sensitivity to sorafenib-induced apoptosis in MOLM14 cells after 48 hours of treatment with E-selectin inhibitor Compound B or E-selectin/CXCR4 inhibitor Compound A.

FIG. 4A is a diagram illustrating establishment of a PDX model in NSG mice by injecting FLT3-ITD mutated AML patient cells (i.v.) after 250 cGy irradiation.

FIG. 4B is a chart showing Kaplan-Meier estimated survival curves for mice receiving Compound A alone, sorafenib alone, or a combination.

FIG. 4C is a diagram showing immunofluorescence images of anti-human CD45 antibody-stained paraffin sections to assess leukemia cell infiltration.

FIG. 4D is a chart showing leukemia cell infiltration measured by anti-CD45 antibody staining for mice receiving Compound A alone, sorafenib alone, or a combination.

FIG. 5A is a diagram showing profiling of cytokine/chemokine by Human Cytokine Antibody Array after co-culture of MOLM14 cells with HS-27A/HEBC-5i in the presence of Compound A and/or sorafenib for 24 h in hypoxia conditions.

FIG. 5B is a diagram showing levels of hematopoiesis-related cytokines and chemokines after co-culture of MOLM14 cells with HS-27A/HEBC-5i in the presence of Compound A and/or sorafenib for 24 hours in hypoxia conditions.

FIG. 5C is a diagram showing microscopic images of bone marrow samples after 53 days of drug treatment stained with stained with H&E or IF using anti-mouse CD41 and CD13 for evaluating megakaryocytes and myelocytes.

FIG. 5D is a chart showing semi-quantitative counts of hematopoietic cells. Error bars present the standard deviation for the mean counts from four random microscopic fields.

FIG. 6 is a chart showing flow cytometry measurements of hCD45+ cells in the bone marrow of MOLM14-engrafted leukemia mice.

FIG. 7 is a chart showing intravital 2-photon microscopy measurements of AML cell velocity in bone marrow after IV infusion of Compound A. Whiskers represent 95% confidence intervals.

FIG. 8 is a diagram showing microscopic images of in vivo calvarial bone marrow cavities four hours post-administration of vehicle (left) or Compound A (right). Images include fluorescent labeling of T cells (hCD2-DsRed), myeloid cells (CD11c-EYFP), osteoblasts (Col2.3-GFP), and bone collagen (SHG).

### DEFINITIONS

Unless otherwise defined, all technical and scientific terms used herein have the same meaning as commonly understood by one of ordinary skill in the art to which this disclosure belongs. All references cited herein are incorporated by reference in their entireties. To the extent terms or discussion in references conflict with this disclosure, the latter shall control.

Whenever a term in the specification is identified as a range (e.g., C₁₋₄ alkyl) or “ranging from”, the range independently discloses and includes each element of the range. As a non-limiting example, C₁₋₄ alkyl groups include, independently, C₁ alkyl groups, C₂ alkyl groups, C₃ alkyl groups, and C₄ alkyl groups. As another non-limiting example, “n is an integer ranging from 0 to 2” includes, independently, 0, 1, and 2.

As used herein, the singular forms of a word also include the plural form of the word, unless the context clearly dictates otherwise. For example, as used herein, “a” or “an” entity refers to one or more of that entity, e.g., “a compound” refers to one or more compounds or at least one compound unless stated otherwise. As such, the terms “a” (or “an”), “one or more”, and “at least one” are used interchangeably herein. For example, the term “at least one C₁₋₄ alkyl group” refers to one or more C₁₋₄ alkyl groups, such as one C₁₋₄ alkyl group, two C₁₋₄ alkyl groups, etc.

The term “or” means “and/or” unless the specific context indicates otherwise.

The term “alkyl” includes saturated straight, branched, and cyclic (also identified as cycloalkyl), primary, secondary, and tertiary hydrocarbon groups. Non-limiting examples of alkyl groups include methyl, ethyl, propyl, isopropyl, cyclopropyl, butyl, secbutyl, isobutyl, tertbutyl, cyclobutyl, 1-methylbutyl, 1,1-dimethylpropyl, pentyl, cyclopentyl, isopentyl, neopentyl, cyclopentyl, hexyl, isohexyl, and cyclohexyl. Unless stated otherwise specifically in the specification, an alkyl group may be optionally substituted. Non-limiting examples of substituted alkyl groups include deuterated alkyl groups such as, e.g., CD3 and CD2CD3.

The term “alkenyl” includes straight, branched, and cyclic hydrocarbon groups comprising at least one double bond. The double bond of an alkenyl group can be unconjugated or conjugated with another unsaturated group. Non-limiting examples of alkenyl groups include vinyl, allyl, butenyl, pentenyl, hexenyl, butadienyl, pentadienyl, hexadienyl, 2-ethylhexenyl, and cyclopent-1-en-1-yl. Unless stated otherwise specifically in the specification, an alkenyl group may be optionally substituted.

The term “alkynyl” includes straight and branched hydrocarbon groups comprising at least one triple bond. The triple bond of an alkynyl group can be unconjugated or conjugated with another unsaturated group. Non-limiting examples of alkynyl groups include ethynyl, propynyl, butynyl, pentynyl, and hexynyl. Unless stated otherwise specifically in the specification, an alkynyl group may be optionally substituted.

The term “aryl” includes hydrocarbon ring system groups comprising at least 6 carbon atoms and at least one aromatic ring. The aryl group may be a monocyclic, bicyclic, tricyclic, or tetracyclic ring system, which may include fused or bridged ring systems. Non-limiting examples of aryl groups include aryl groups derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group may be optionally substituted.

The term “halo” or “halogen” includes fluoro, chloro, bromo, and iodo.

The term “haloalkyl” includes alkyl groups, as defined herein, substituted by at least one halogen, as defined herein. Non-limiting examples of haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl. A “fluoroalkyl” is a haloalkyl wherein at least one halogen is fluoro. Unless stated otherwise specifically in the specification, a haloalkyl group may be optionally substituted.

The term “haloalkenyl” includes alkenyl groups, as defined herein, substituted by at least one halogen, as defined herein. Non-limiting examples of haloalkenyl groups include fluoroethenyl, 1,2-difluoroethenyl, 3-bromo-2-fluoropropenyl, and 1,2-dibromoethenyl. A “fluoroalkenyl” is a haloalkenyl substituted with at least one fluoro group. Unless stated otherwise specifically in the specification, a haloalkenyl group may be optionally substituted.

The term “haloalkynyl” includes alkynyl groups, as defined herein, substituted by at least one halogen, as defined herein. Non-limiting examples include fluoroethynyl, 1,2-difluoroethynyl, 3-bromo-2-fluoropropynyl, and 1,2-dibromoethynyl. A “fluoroalkynyl” is a haloalkynyl wherein at least one halogen is fluoro. Unless stated otherwise specifically in the specification, a haloalkynyl group may be optionally substituted.

The term “heterocyclyl” or “heterocyclic ring” includes 3- to 24-membered saturated or partially unsaturated non-aromatic ring groups comprising 2 to 23 ring carbon atoms and 1 to 8 ring heteroatom(s) each independently chosen from N, O, and S. Unless stated otherwise specifically in the specification, the heterocyclyl groups may be monocyclic, bicyclic, tricyclic or tetracyclic ring systems, which may include fused or bridged ring systems, and may be partially or fully saturated; any nitrogen, carbon or sulfur atom(s) in the heterocyclyl group may be optionally oxidized; any nitrogen atom in the heterocyclyl group may be optionally quaternized; and the heterocyclyl group. Non-limiting examples of heterocyclic ring include dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

The term “heteroaryl” includes 5- to 14-membered ring groups comprising 1 to 13 ring carbon atoms and 1 to 6 ring heteroatom(s) each independently chosen from N, O, and S, and at least one aromatic ring. Unless stated otherwise specifically in the specification, the heteroaryl group may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Non-limiting examples include azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, I-oxidopyridazinyl, 1-phenyl-IH-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group may be optionally substituted.

Unless stated otherwise specifically in the specification, substituents may be optionally substituted.

The term “substituted” includes the situation where, in any of the above groups, at least one hydrogen atom is replaced by a non-hydrogen atom such as, for example, a deuterium atom; a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also includes the situation where, in any of the above groups, at least one hydrogen atom is replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles.

The terms “acute myeloid leukemia,” “acute myelogenous leukemia,” “acute myeloblastic leukemia,” “acute granulocytic leukemia,” and “acute nonlymphocytic leukemia,” and “AML” are used interchangeably and as used herein, refer to a cancer of the bone marrow characterized by abnormal proliferation of myeloid stem cells. AML, as used herein, refers to any or all known subtypes of the disease, including but not limited to, subtypes classified by the World Health Organization (WHO) 2016 classification of AML, e.g., AML with myelodysplasia-related changes or myeloid sarcoma, and the French-American-British (FAB) classification system, e.g., M0 (acute myeloblastic leukemia, minimally differentiated) or M1 (acute myeloblastic leukemia, without maturation) (Falini et al., 2010; Lee et al., 1987).

As used herein, “administration” of a compound to a patient refers to any route (e.g., oral delivery) of introducing or delivering the API to the patient. Administration includes self-administration and the administration by another.

As used herein, the terms “blasts” and “blast cells” are used interchangeably to refer to undifferentiated, precursor blood stem cells. As used herein, the term “blast count” refers to the number of blast cells in a sample.

As used herein, an “effective amount” or “effective dose” refers to an amount of a compound that treats, upon single or multiple dose administration, a patient suffering from a condition. An effective amount can be determined by the attending diagnostician through the use of known techniques and by observing results obtained under analogous circumstances. In determining the effective amount, a number of factors are considered by the attending diagnostician, including, but not limited to: the patient's size, age, and general health; the specific condition, disorder, or disease involved; the degree of or involvement or the severity of the condition, disorder, or disease, the response of the individual patient; the particular compound administered; the mode of administration; the bioavailability characteristics of the preparation administered; the dose regimen selected; the use of concomitant medication; and other relevant circumstances.

As used herein, the term “E-selectin antagonist” includes antagonists of E-selectin only, as well as antagonists of E-selectin and either P-selectin or L-selectin, and antagonists of E-selectin, P-selectin, and L-selectin. The terms “E-selectin antagonist” and “E-selectin inhibitor” are used interchangeably herein.

In some embodiments, the E-selectin antagonist inhibits an activity of E-selectin or inhibits the binding of E-selectin to one or more E-selectin ligands (which in turn may inhibit a biological activity of E-selectin).

E-selectin antagonists include the glycomimetic compounds described herein. E-selectin antagonists also include antibodies, polypeptides, peptides, peptidomimetics, and aptamers which bind at or near the binding site on E-selectin to inhibit E-selectin interaction with sialyl Le^{a} (sLe^{a}) or sialyl Le^{x} (sLe^{x}).

Further disclosure regarding E-selectin antagonists suitable for the disclosed methods (e.g., compounds and compositions) may be found in U.S. Pat. No. 9,254,322, issued Feb. 9, 2016, and U.S. Pat. No. 9,486,497, issued Nov. 8, 2016, which are hereby incorporated by reference. In some embodiments, the E-selectin antagonist is chosen from E-selectin antagonists disclosed in U.S. Pat. No. 9,109,002, issued Aug. 18, 2015, which is hereby incorporated by reference. In some embodiments, the E-selectin antagonist is chosen from heterobifunctional antagonists disclosed in U.S. Pat. No. 8,410,066, issued Apr. 2, 2013, and U.S. Pat. No. 10,519,181, issued Dec. 31, 2019, which are hereby incorporated by reference. Further disclosure regarding E-selectin antagonists suitable for the disclosed methods and compounds may be found in U.S. Publication No. US2019/0233458, published Aug. 1, 2019, WO2019/133878, published Jul. 4, 2019, WO 2020/139962, published Jul. 2, 2020, WO 2020/219419, published Oct. 29, 2020, and WO 2020/219417, published Oct. 29, 2020, which are hereby incorporated by reference.

In some embodiments, the E-selectin antagonists suitable for the disclosed methods include pan-selectin antagonists.

As used herein, “CXCR4 receptor inhibitor” and “CXCR4 chemokine receptor inhibitor” are used interchangeably and mean an agent inhibits the binding of the chemokine SDF-I to an SDF-I ligand (e.g., prevents the binding of SDF-I to CXCR4). Such inhibitors will typically prevent the binding of stromal derived factor-1 (SDF-1) to a CXCR4 receptor. Non-limiting example of CXCR4 chemokine receptor inhibitors include AMD-3100 (Hendrix et al., 2000); ALX40-4C (Doranz et al., 2001); and T134 (Arakaki et al., 1999). These examples include a small organic molecule and amino acid-based molecules, such as the T22 peptide. AMD-3100 is a bicyclam. Each of the two cyclam rings is attached to the same phenyl ring (each cyclam ring is para to the other) via a methylene group.

In some embodiments, CXCR4 receptor inhibitors may be chosen from peptides, diketopiperazine mimetics, bicyclams, tetrahydroquinolines, thiazolylisothiourea derivatives, and benzodiazepines. In some embodiments, CXCR4 receptor inhibitors may be chosen from AMD-3100, ALX40-4C, T134, and T22 peptide.

Heterobifunctional compounds for inhibition of E-selectin and the CXCR4 chemokine receptor comprising E-selectin inhibitor-Linker-CXCR4 chemokine receptor inhibitor are also known in the art. Non-limiting examples are disclosed, for example, in U.S. Pat. No. 8,410,066.

As used herein, an amount expressed in terms of “mg of at least one compound chosen from [X] and pharmaceutically acceptable salts thereof” is based on the total weight of the free base of [X] present, in the form of the free base and/or one or more pharmaceutically acceptable salts of [X]. One of ordinary skill in the art would understand the amount of pharmaceutically acceptable derivative, such as a pharmaceutically acceptable salt, that is equivalent to the daily dosages and individual doses of a compound described herein. That is, for example, given the disclosure above of a fixed daily dose of 1600 mg of Compound A, one of ordinary skill in the art would understand how to determine an equivalent fixed daily dose of a pharmaceutically acceptable salt of Compound A.

As used herein, the term “increase” refers to altering positively by at least 1%, including, but not limited to, altering positively by at least 5% (e.g., by 5%), altering positively by at least 10% (e.g., 10%), altering positively by at least 25% (e.g., by 25%), altering positively by at least 30% (e.g., by 30%), altering positively by at least 50% (e.g., by 50%), altering positively by at least 75% (e.g., by 75%), or altering positively by 100%, altering positively by 5% to 10%, altering positively by 5% to 15%, altering positively by 5% to 25%, etc.

As used herein, the term “modulate” refers to altering positively or negatively. Non-limiting example modulations include an at least 1% (e.g., a 1%) change, an at least a 2% (e.g., 2%) change, an at least a 5% (e.g., 5%) change, an at least a 10% (e.g., a 10%) change, an at least a 25% (e.g., 25%) change, an at least a 50% (e.g., 50%) change, an at least a 75% (e.g., a 75%) change, a 100% change, a 5% to 10% change, a 5% to 15% change, a 5% to 25% change, etc.

As used herein, the terms “patient” and “subject” are used interchangeably. In some embodiments, the patient or subject is a mammal. In some embodiments, the patient or subject is a human.

As used herein, the term “pharmaceutical composition” refers to a mixture or a combination of at least one active pharmaceutical ingredient and at least one pharmaceutically acceptable excipient. Pharmaceutical compositions may be administered in any manner appropriate to the disease or disorder to be treated as determined by persons of ordinary skill in the medical arts. An appropriate dose and a suitable duration and frequency of administration will be determined by such factors as discussed herein, including the condition of the patient, the type and severity of the patient's disease, the particular form of the active ingredient, and the method of administration. In general, an appropriate dose (or effective dose) and treatment regimen provides the pharmaceutical composition in an amount sufficient to provide therapeutic and/or prophylactic benefit (for example, an improved clinical outcome, such as more frequent complete or partial remissions, or longer disease-free and/or overall survival, or a lessening of symptom severity or other benefit as described in detail herein).

The pharmaceutical compositions described herein may be administered to a subject in need thereof by any of several routes that can effectively deliver an effective amount of the compound. In some embodiments, the pharmaceutical composition is administered parenterally. Non-limiting suitable routes of parenteral administration include subcutaneous, intravenous, intramuscular, intrasternal, intracavernous, intrameatal, and intraurethral injection and/or infusion. In some embodiments, the pharmaceutical composition is administered intravenously (IV). Non-limiting suitable routes of IV administration include via a peripheral line, a central catheter, and a peripherally inserted central line catheter (PICC). In some embodiments, the pharmaceutical composition is administered subcutaneously.

The pharmaceutical compositions described herein may be sterile aqueous or sterile non-aqueous solutions, suspensions, or emulsions, and may additionally comprise at least one pharmaceutically acceptable excipient or diluent (i.e., a non-toxic material that does not interfere with the activity of the active ingredient). Such compositions may be in the form of a solid, liquid, or gas (aerosol). A liquid pharmaceutical composition may include, for example, at least one the following: a sterile diluent such as water for injection; saline solution (e.g., physiological saline); Ringer's solution; isotonic sodium chloride; fixed oils that may serve as the solvent or suspending medium; polyethylene glycols; glycerin; propylene glycol or other solvents; antibacterial agents; antioxidants; chelating agents; buffers and agents for the adjustment of tonicity, such as, e.g., sodium chloride or dextrose. A parenteral preparation may be enclosed in ampoules, disposable syringes or multiple dose vials made of glass or plastic. In some embodiments, the pharmaceutical composition comprises physiological saline. In some embodiments, the pharmaceutical composition is an injectable pharmaceutical composition, and in some embodiments, the injectable pharmaceutical composition is sterile.

In some embodiments, a pharmaceutical composition is a solid pharmaceutical composition. In some embodiments, a pharmaceutical composition is a pharmaceutical composition for oral administration. In some embodiments, a pharmaceutical composition is a single dosage unit form. In some embodiments, a pharmaceutical composition is a multiple dosage unit form. In some embodiments, a pharmaceutical composition is a tablet composition. In some embodiments, a pharmaceutical composition is a capsule composition.

In some embodiments, a pharmaceutical composition is formulated as a liquid. In some embodiments, a pharmaceutical composition is formulated as a liquid for intravenous administration. In some embodiments, a pharmaceutical composition is formulated as a liquid for parenteral administration. In some embodiments, a pharmaceutical composition is formulated as a liquid for subcutaneous (subQ) administration. In some embodiments, a pharmaceutical composition is formulated as a liquid for intramuscular (IM) administration. In some embodiments, a pharmaceutical composition is formulated as a liquid for intraosseous administration.

As used herein, a “pharmaceutically acceptable excipient” refers to a carrier or an excipient that is useful in preparing a pharmaceutical composition. For example, a pharmaceutically acceptable excipient is generally safe and includes carriers and excipients that are generally considered acceptable for mammalian pharmaceutical use. As a non-limiting example, pharmaceutically acceptable excipients may be solid, semi-solid, or liquid materials which in the aggregate can serve as a vehicle or medium for the active ingredient. Some examples of pharmaceutically acceptable excipients are found in Remington's Pharmaceutical Sciences and the Handbook of Pharmaceutical Excipients and include diluents, vehicles, carriers, ointment bases, binders, disintegrates, lubricants, glidants, sweetening agents, flavoring agents, gel bases, sustained release matrices, stabilizing agents, preservatives, solvents, suspending agents, buffers, emulsifiers, dyes, propellants, coating agents, and others.

In general, the type of excipient or diluent is selected based on the mode of administration, as well as the chemical composition of the active ingredient(s). As a non-limiting example, pharmaceutical compositions for parenteral administration may further comprise one or more of water, saline, alcohols, fats, waxes, and buffers.

As used herein, the term “pharmaceutically acceptable salts” includes both acid and base addition salts. Non-limiting examples of pharmaceutically acceptable acid addition salts include chlorides, bromides, sulfates, nitrates, phosphates, sulfonates, methane sulfonates, formates, tartrates, maleates, citrates, benzoates, salicylates, and ascorbates. Non-limiting examples of pharmaceutically acceptable base addition salts include sodium, potassium, lithium, ammonium (substituted and unsubstituted), calcium, magnesium, iron, zinc, copper, manganese, and aluminum salts. Pharmaceutically acceptable salts may, for example, be obtained using standard procedures well known in the field of pharmaceuticals.

As used herein, the term “prodrug” includes compounds that may be converted, for example, under physiological conditions or by solvolysis, to a biologically active compound described herein. Thus, the term “prodrug” includes metabolic precursors of compounds described herein that are pharmaceutically acceptable. A discussion of prodrugs can be found, for example, in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987. The term “prodrug” also includes covalently bonded carriers that release the active compound(s) as described herein in vivo when such prodrug is administered to a subject. Non-limiting examples of prodrugs include ester and amide derivatives of hydroxy, carboxy, mercapto and amino functional groups in the compounds described herein.

As used herein, the term “reduce” refers to altering negatively by at least 1% including, but not limited to, altering negatively by at least 5% (e.g., by 5%), altering negatively by at least 10% (e.g., by 10%), altering negatively by at least 25% (e.g., by 25%), altering negatively by at least 30% (e.g., by 30%), altering negatively by at least 50% (e.g., by 50%4), altering negatively by at least 75% (e.g., by 75%), altering negatively by 100%, altering negatively by 5% to 10%, altering negatively by 5% to 15%, altering negatively by 5% to 25%, etc.

As used herein, the term “treat,” “treating,” or “treatment,” when used in connection with a disorder or condition, includes any effect, e.g., lessening, reducing, modulating, ameliorating, or eliminating, that results in the improvement of the disorder or condition. The effect can be prophylactic in terms of completely or partially preventing a disease or symptom thereof from occurring in the first place and/or can be therapeutic in terms of a partial or complete cure for a disease and/or adverse effects attributable to the disease. As a non-limiting example, the term “treatment” and the like, as used herein, encompasses any treatment of cancers, such as, e.g., AML or any of its subtypes and related hematologic cancers in a mammal, such as, e.g., in a human, and includes: (a) preventing the disease from occurring in a subject, e.g., a subject identified as predisposed to the disease or at risk of acquiring the disease but has not yet been diagnosed as having it; (b) delaying onset or progression of the disease, e.g., as compared to the anticipated onset or progression of the disease in the absence of treatment; (c) inhibiting the disease, i.e., arresting its development; and/or (d) relieving the disease, i.e., causing regression of the disease. Improvements in or lessening the severity of any symptom of the disorder or condition can be readily assessed according to standard methods and techniques known in the art.

In some embodiments, “treating” refers to administering, e.g., subcutaneously, an effective dose, or effective multiple doses of a composition, e.g., a composition comprising at least one FLT3 inhibitor and at least one inhibitor chosen from E-selectin inhibitors, CXCR4 inhibitors, and heterobifunctional inhibitors of E-selectin and CXCR4 as disclosed herein to an animal (including a human being) suspected of suffering or already suffering from AML or another related cancer.

In some embodiments, an effective dose is a dose that partially or fully alleviates (i.e., eliminates or reduces) at least one symptom associated with the disorder/disease state being treated, that slows, delays, or prevents onset or progression to a disorder/disease state, that slows, delays, or prevents progression of a disorder/disease state, that diminishes the extent of disease, that reverse one or more symptoms, that results in remission (partial or total) of disease, and/or that prolongs survival. Examples of disease states contemplated for treatment are set out herein. In some embodiments, the patient currently has cancer, was once treated for cancer and is in remission, or is at risk of relapsing after treatment for the cancer.

In some embodiments, “treating” can also refer to reducing, eliminating, or at least partially arresting, as well as to exerting any beneficial effect, on one or more symptoms of the disease and/or associated with the disease and/or its complications.

This application contemplates all the isomers of the compounds disclosed herein. “Isomer” as used herein includes optical isomers (such as stereoisomers, e.g., enantiomers and diastereoisomers), geometric isomers (such as Z (zusammen) or E (entgegen) isomers), and tautomers. The present disclosure includes within its scope all the possible geometric isomers, e.g., Z and E isomers (cis and trans isomers), of the compounds as well as all the possible optical isomers, e.g., diastereomers and enantiomers, of the compounds. Furthermore, the present disclosure includes in its scope both the individual isomers and any mixtures thereof, e.g., racemic mixtures. The individual isomers may be obtained using the corresponding isomeric forms of the starting material or they may be separated after the preparation of the end compound according to conventional separation methods. For the separation of optical isomers, e.g., enantiomers, from the mixture thereof conventional resolution methods, e.g., fractional crystallization, may be used.

The present disclosure includes within its scope all possible tautomers. Furthermore, the present disclosure includes in its scope both the individual tautomers and any mixtures thereof. Each compound disclosed herein includes within its scope all possible tautomeric forms. Furthermore, each compound disclosed herein includes within its scope both the individual tautomeric forms and any mixtures thereof. With respect to the methods, uses and compositions of the present application, reference to a compound or compounds is intended to encompass that compound in each of its possible isomeric forms and mixtures thereof. Where a compound of the present application is depicted in one tautomeric form, that depicted structure is intended to encompass all other tautomeric forms.

### Non-Limiting Example Embodiments:

Without limitation, some embodiments of this disclosure include:

1. A method of treating a cancer in a subject in need thereof comprising administering to the subject at least one FLT3 inhibitor and at least one inhibitor chosen from E-selectin inhibitors, CXCR4 inhibitors, and heterobifunctional inhibitors of E-selectin and CXCR4.

2. A method of enhancing maintenance of normal hematopoiesis in bone marrow in a subject in need thereof comprising administering to the subject at least one FLT3 inhibitor and at least one inhibitor chosen from E-selectin inhibitors, CXCR4 inhibitors, and heterobifunctional inhibitors of E-selectin and CXCR4.

3. A method of mobilizing leukemia blasts in a subject in need thereof comprising administering to the subject at least one FLT3 inhibitor and at least one inhibitor chosen from E-selectin inhibitors, CXCR4 inhibitors, and heterobifunctional inhibitors of E-selectin and CXCR4.

4. The method according to any one of Embodiments 1-3, wherein the subject is a human.

5. The method according to any one of Embodiments 1-4, wherein the subject is a cancer patient.

6. The method according to any one of Embodiments 1-5, wherein the subject is a relapsed cancer patient.

7. The method according to any one of Embodiments 1 or 4-6, wherein the administration of the at least one FLT3 inhibitor and at least one inhibitor chosen from E-selectin inhibitors, CXCR4 inhibitors, and heterobifunctional inhibitors of E-selectin and CXCR4 extends the number of days the subject is in remission, reduces the number of days until remission, inhibits the metastasis of cancer cells, and/or improves survival.

8. The method according to any one of Embodiments 1-7, wherein the subject is receiving, has received, or will receive chemotherapy and/or radiotherapy.

9. The method according to any one of Embodiments 1-8, wherein the subject is receiving, has received, or will receive two or more chemotherapeutic agents (such as, e.g., mitoxantrone, etoposide, and cytarabine or fludarabine, cytarabine, and idarubicin).

10. The method according to any one of Embodiments 1-9, wherein the subject is receiving, has received, or will receive velafermin, palifermin, thalidomide, and/or a thalidomide derivative.

11. The method according to any one of Embodiments 1-10, wherein the subject is receiving, has received, or will receive MMP inhibitors, inflammatory cytokine inhibitors, mast cell inhibitors, NSAIDs, NO inhibitors, MDM2 inhibitors, or antimicrobial compounds.

12. The method according to any one of Embodiments 1 or 4-11, wherein the cancer is chosen from liquid cancers.

13. The method according to any one of Embodiments 1 or 4-11, wherein the cancer is chosen from solid cancers.

14. The method according to any one of Embodiments 1 or 4-11, wherein the cancer is chosen from colorectal cancer, liver cancer, gastric cancer, lung cancer, brain cancer, kidney cancer, bladder cancer, thyroid cancer, prostate cancer, ovarian cancer, cervical cancer, uterine cancer, endometrial cancer, breast cancer, pancreatic cancer, leukemia, lymphoma, myeloma, melanoma, kidney chromophobe carcinoma, adrenocortical carcinoma, bladder urothelial carcinoma, thymoma, testicular germ cell tumors, and head and neck squamous cell carcinoma.

15. The method according to any one of Embodiments 1, 4-11, or 14, wherein the cancer is chosen from melanoma, leukemia, kidney chromophobe carcinoma, adrenocortical carcinoma, bladder urothelial carcinoma, lymphoma, thymoma, testicular germ cell tumors, and head and neck squamous cell carcinoma.

16. The method according to any one of Embodiments 1, 4-11, 14, or 15, wherein the leukemia is chosen from acute myeloid leukemia, acute lymphocytic leukemia, chronic lymphocytic leukemia, and chronic myelogenous leukemia.

17. The method according to any one of Embodiments 1, 4-11, 14, or 15, wherein the lymphoma is chosen from non-Hodgkin's lymphoma and Hodgkin's lymphoma.

18. The method according to any one of Embodiments 1, 4-11, 14, or 15, wherein the myeloma is multiple myeloma.

19. The method according to any one of Embodiments 1, 4-11, 14, or 15, wherein the melanoma is chosen from uveal melanoma and skin melanoma.

20. The method according to any one of Embodiments 1 or 4-11, wherein the cancer is chosen from FLT3 mutated cancers.

21. The method according to any one of Embodiments 1 or 4-12, wherein the cancer is chosen from FLT3-ITD mutated cancers.

22. The method according to any one of Embodiments 1, 4-12, 14-16, 20, or 21, wherein the cancer is AML.

23. The method according to any one of Embodiments 1, 4-12, 14-16, or 20-22, wherein the cancer is relapsed/refractory AML.

24. The method according to any one of Embodiments 1, 4-12, 14-16, or 20-23, wherein the cancer is FLT3-ITD mutated AML.

25. The method according to any one of Embodiments 1-24, wherein the subject possesses one or more mutational alterations of FLT3.

26. The method according to Embodiment 25, wherein the mutational alterations are chosen from internal tandem duplications and missense mutations within the tyrosine kinase domain activation loop of FLT3.

27. The method according to Embodiment 25 or 26, wherein the mutational alterations are chosen from internal tandem duplications within the tyrosine kinase domain activation loop of FLT3.

28. The method according to Embodiment 25 or 26, wherein the mutational alterations are chosen from missense mutations within the tyrosine kinase domain activation loop of FLT3.

29. The method according to any one of Embodiments 1-28, wherein the blast cells in the subject have an increased gene expression level of FUT7 relative to a control sample from a non-cancer subject, a newly diagnosed cancer subject, or a subject having the same cancer as the patient.

30. The method according to any one of Embodiments 1-29, comprising administering to the subject at least one FLT3 inhibitor and at least one E-selectin inhibitor.

31. The method according to any one of Embodiments 1-29, comprising administering to the subject at least one FLT3 inhibitor and at least one CXCR4 inhibitor.

32. The method according to any one of Embodiments 1-29, comprising administering to the subject at least one FLT3 inhibitor and at least one heterobifunctional inhibitor of E-selectin and CXCR4.

33. The method according to any one of Embodiments 1-29, comprising administering to the subject at least one FLT3 inhibitor, at least one E-selectin inhibitor, and at least one CXCR4 inhibitor.

34. The method according to any one of Embodiments 1-29, wherein the at least one inhibitor is chosen from Compound A, Compound B, Compound C, Compound D, Compound E, and pharmaceutically acceptable salts of any of the foregoing.

35. The method according to any one of Embodiments 1-29, wherein the at least one compound is chosen from compounds of Formula (I), (Ia), (II), (IIa), (III), (IIIa), (IV), (V), (IVa/Va), (IVb/Vb), (VI), (VII), and (VIII) and pharmaceutically acceptable salts of any of the foregoing.

36. The method according to any one of Embodiments 1-29, wherein the at least one inhibitor is chosen from

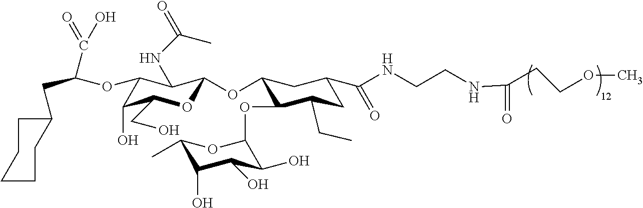

and pharmaceutically acceptable salts thereof.

37. The method according to any one of Embodiments 1-29, wherein the at least one inhibitor is

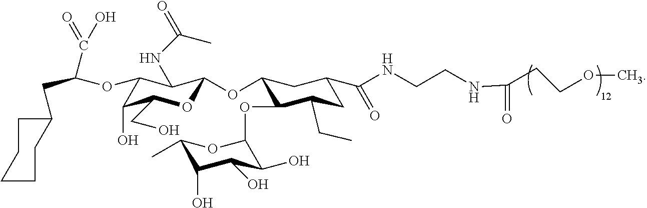

38. The method according to any one of Embodiments 1-29, wherein the at least one inhibitor is chosen from

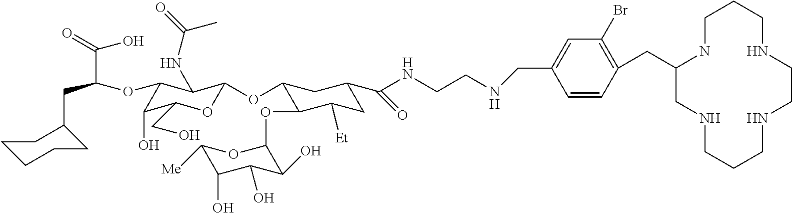 [SMILES: CCC1CC(C(=O)NCCNCc2ccc(CC3CNCCCNCCNCCCN3)c(Br)c2)CC(OC2OC(CO)C(O)C(O[C@@H](CC3CCCCC3)C(=O)O)C2NC(C)=O)C1OC1OC(C)C(O)C(O)C1O]

and pharmaceutically acceptable salts thereof.

39. The method according to any one of Embodiments 1-29, wherein the at least one inhibitor is

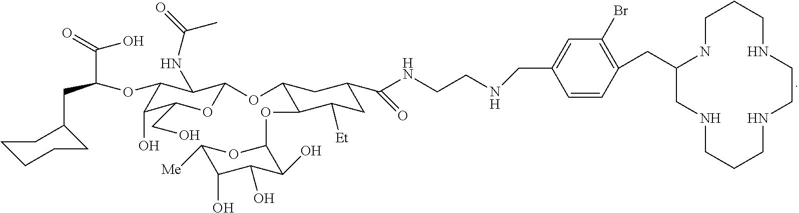 [SMILES: CCC1CC(C(=O)NCCNCc2ccc(CC3CNCCCNCCNCCCN3)c(Br)c2)CC(OC2OC(CO)C(O)C(O[C@@H](CC3CCCCC3)C(=O)O)C2NC(C)=O)C1OC1OC(C)C(O)C(O)C1O]

40. The method according to any one of Embodiments 1-29 or 34-39, wherein the method comprises administering a dose in the range of 5 mg/kg to 100 mg/kg (such as, e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one inhibitor (such as, e.g., at least one E-selectin inhibitor, at least one CXCR4 inhibitor, or at least one heterobifunctional inhibitor of E-selectin and CXCR4).

41. The method according to any one of Embodiments 1-29 or 34-39, wherein the method comprises administering a fixed dose of 20 mg to 4000 mg (such as, e.g., 20 mg, 30 mg, 40 mg, 50 mg, 60 mg, 70 mg, 80 mg, 90 mg, 100 mg, 125 mg, 150 mg, 175 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, e.g., 800 mg to 3200 mg per day, 1000 mg to 2000 mg per day) per day of the at least one inhibitor (such as, e.g., at least one E-selectin inhibitor, at least one CXCR4 inhibitor, or at least one heterobifunctional inhibitor of E-selectin and CXCR4).

42. The method according to any one of Embodiments 1-41, wherein the method comprises administering a dose in the range of 0.5 mg/kg to 100 mg/kg (such as, e.g., 0.5 mg/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one FLT3 inhibitor.

43. The method according to any one of Embodiments 1-41, wherein the method comprises administering a fixed dose of 20 mg to 4000 mg per day mg (such as, e.g., 20 mg, 30 mg, 40 mg, 50 mg, 60 mg, 70 mg, 80 mg, 90 mg, 100 mg, 125 mg, 150 mg, 175 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, e.g., 800 mg to 3200 mg per day, 1000 mg to 2000 mg per day) of the at least FLT3 inhibitor.

44. The method according to any one of Embodiments 1-43, wherein the at least one FLT3 inhibitor is chosen from first generation multi-kinase inhibitors and next generation inhibitors.

45. The method according to any one of Embodiments 1-44, wherein the at least one FLT3 inhibitor is chosen from sorafenib, lestaurtinib, midostaurin, quizartinib, crenolanib, gilteritinib, and pharmaceutically acceptable salts of any of the foregoing.

46. The method according to any one of Embodiments 1-45, wherein the at least one FLT3 inhibitor is sorafenib.

47. The method according to Embodiment 46, wherein the method comprises administering a fixed dose of 400 mg twice daily of sorafenib.

48. The method according to any one of Embodiments 1-45, wherein the at least one FLT3 inhibitor is quizartinib.

49. The method according to Embodiment 48, wherein the method comprises administering a fixed dose of 30 mg, 60 mg, or 90 mg once per day of quizartinib.

Some embodiments of the disclosure relate to a method of treating a cancer patient, comprising:
1. obtaining or having obtained a biological sample comprising blast cells from the cancer patient;
2. performing or having performed an assay on the biological sample to determine the gene expression level of FUT7; and
3. if the blast cells in the sample have an increased gene expression level of FUT7 relative to a control sample from a non-cancer subject, a newly-diagnosed cancer subject, or a subject having the same cancer as the patient, or
if at least 10% of the blast cells in the sample express FUT7, then

administering to the cancer patient at least one FLT3 inhibitor and at least one inhibitor chosen from E-selectin inhibitors, CXCR4 inhibitors, and heterobifunctional inhibitors of E-selectin and CXCR4.

Methods to measure gene expression levels are known to persons of skill in the art. Gene expression may be measured by the number of mRNA transcripts or the amount of protein expressed. Exemplary methods to measure the amount of mRNA include, but are not limited to, Sanger sequencing, high throughput sequencing, quantitative polymerase chain reaction (qPCR), reverse transcriptase qPCR (RT-qPCR), RNA sequencing, microarray analysis, and Northern blots. In some embodiments, gene expression level is measured by RNA-seq. In some embodiments, gene expression level is measured by high coverage mRNA sequencing.

In some embodiments, gene expression level is measured by the amount of mRNA. In some embodiments, the method comprises measuring the amount of mRNA encoding FUT7.

Gene expression may also be measured by the amount of protein in a patient sample. Exemplary methods to measure the amount of protein include, but are not limited, to immunostaining, immunohistochemistry, affinity purification, mass spectrometry, Western blotting, and enzyme-linked immunosorbent assay (ELISA).

In some embodiments, gene expression level is measured by the amount of protein in a patient sample. In some embodiments, the method comprises measuring the amount of FUT7 protein in a patient sample.

In some embodiments, the method further comprises determining the presence of one or more mutational alterations of FLT3. In some embodiments, the mutational alterations are chosen from internal tandem duplications and missense mutations within the tyrosine kinase domain activation loop of FLT3.

In some embodiments, the one or more diagnostic assays may comprise assays to detect expression of E-selectin ligand on the surface of FLT3 AML cells, and may include flow analysis, flow cytometry, or immunohistology using the appropriate reagents. In some embodiments, the reagents for immunohistology may include a HECA-452-FITC monoclonal antibody, or similar reagents. In other embodiments, the reagents for immunohistology may include an E-selectin/hIg chimera/PE, or similar reagents.

In some embodiments, the cancer patient is a relapsed cancer patient.

In some embodiments, the cancer patient has a cancer chosen from solid cancers and liquid cancers.

In some embodiments, the cancer patient has one or more cancers chosen from colorectal cancer, liver cancer, gastric cancer, lung cancer, brain cancer, kidney cancer, bladder cancer, thyroid cancer, prostate cancer, ovarian cancer, cervical cancer, uterine cancer, endometrial cancer, breast cancer, pancreatic cancer, leukemia, lymphoma, myeloma, melanoma, kidney chromophobe carcinoma, adrenocortical carcinoma, bladder urothelial carcinoma, thymoma, testicular germ cell tumors, and head and neck squamous cell carcinoma.

In some embodiments, the cancer patient has one or more cancers chosen from melanoma, leukemia, kidney chromophobe carcinoma, adrenocortical carcinoma, bladder urothelial carcinoma, lymphoma, thymoma, testicular germ cell tumors, and head and neck squamous cell carcinoma.

In some embodiments, the leukemia is chosen from acute myeloid leukemia, acute lymphocytic leukemia, chronic lymphocytic leukemia, and chronic myelogenous leukemia.

In some embodiments, the lymphoma is chosen from non-Hodgkin's lymphoma and Hodgkin's lymphoma.

In some embodiments, the myeloma is multiple myeloma.

In some embodiments, the melanoma is chosen from uveal melanoma and skin melanoma.

Some of the embodiments of the present disclosure relate to a method of treating a cancer in a subject in need thereof comprising administering to the subject at least one FLT3 inhibitor and at least one inhibitor chosen from E-selectin inhibitors, CXCR4 inhibitors, and heterobifunctional inhibitors of E-selectin and CXCR4.

Some of the embodiments of the present disclosure relate to a method of enhancing maintenance of normal hematopoiesis in bone marrow in a subject in need thereof comprising administering to the subject at least one FLT3 inhibitor and at least one inhibitor chosen from E-selectin inhibitors, CXCR4 inhibitors, and heterobifunctional inhibitors of E-selectin and CXCR4.

Some of the embodiments of the present disclosure relate to a method of mobilizing leukemia blasts in a subject in need thereof comprising administering to the subject at least one FLT3 inhibitor and at least one inhibitor chosen from E-selectin inhibitors, CXCR4 inhibitors, and heterobifunctional inhibitors of E-selectin and CXCR4.

In some embodiments, the subject possesses one or more mutational alterations of FLT3. In some embodiments, the mutational alterations are chosen from internal tandem duplications and missense mutations within the tyrosine kinase domain activation loop of FLT3.

In some embodiments, blast cells in the subject have an increased gene expression level of FUT7 relative to a control sample from a non-cancer subject, a newly-diagnosed cancer subject, or a subject having the same cancer as the patient.

In some embodiments, the subject is a cancer patient. In some embodiments, the subject is a relapsed cancer patient.

In some embodiments, the subject has a cancer chosen from solid cancers and liquid cancers.

In some embodiments, the subject has one or more cancers chosen from colorectal cancer, liver cancer, gastric cancer, lung cancer, brain cancer, kidney cancer, bladder cancer, thyroid cancer, prostate cancer, ovarian cancer, cervical cancer, uterine cancer, endometrial cancer, breast cancer, pancreatic cancer, leukemia, lymphoma, myeloma, melanoma, kidney chromophobe carcinoma, adrenocortical carcinoma, bladder urothelial carcinoma, thymoma, testicular germ cell tumors, and head and neck squamous cell carcinoma.

In some embodiments, the subject has one or more cancers chosen from melanoma, leukemia, kidney chromophobe carcinoma, adrenocortical carcinoma, bladder urothelial carcinoma, lymphoma, thymoma, testicular germ cell tumors, and head and neck squamous cell carcinoma.

In some embodiments, the leukemia is chosen from acute myeloid leukemia, acute lymphocytic leukemia, chronic lymphocytic leukemia, and chronic myelogenous leukemia.

In some embodiments, the lymphoma is chosen from non-Hodgkin's lymphoma and Hodgkin's lymphoma.

In some embodiments, the myeloma is multiple myeloma.

In some embodiments, the melanoma is chosen from uveal melanoma and skin melanoma.

In some embodiments of a method disclosed herein, the administration of the at least one FLT3 inhibitor and at least one inhibitor chosen from E-selectin inhibitors, CXCR4 inhibitors, and heterobifunctional inhibitors of E-selectin and CXCR4 extends the number of days the subject is in remission, reduces the number of days until remission, inhibits the metastasis of cancer cells, and/or improves survival.

In some embodiments of a method disclosed herein, where the subject has AML, the administration of the at least one FLT3 inhibitor and at least one inhibitor chosen from E-selectin inhibitors, CXCR4 inhibitors, and heterobifunctional inhibitors of E-selectin and CXCR4 extends the number of days the subject is in remission, reduces the number of days until remission, inhibits the metastasis of AML cells, and/or improves survival.

In some embodiments of a method disclosed herein, the subject is receiving, has received, or will receive chemotherapy and/or radiotherapy. In some embodiments, the subject is receiving, has received, or will receive two or more chemotherapeutic agents, such as, e.g., mitoxantrone, etoposide, and cytarabine or fludarabine, cytarabine, and idarubicin. In some embodiments, the subject is receiving, has received, or will receive velafermin, palifermin, thalidomide, and/or a thalidomide derivative.

In some embodiments of a method disclosed herein, the subject is receiving, has received, or will receive MMP inhibitors, inflammatory cytokine inhibitors, mast cell inhibitors, NSAIDs, NO inhibitors, MDM2 inhibitors, or antimicrobial compounds.

In some embodiments of a method disclosed herein, the at least one inhibitor is chosen from

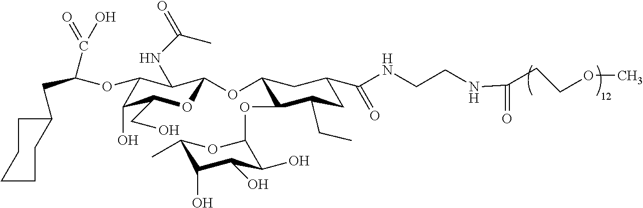

and pharmaceutically acceptable salts thereof.

In some embodiments of a method disclosed herein, the at least one inhibitor is

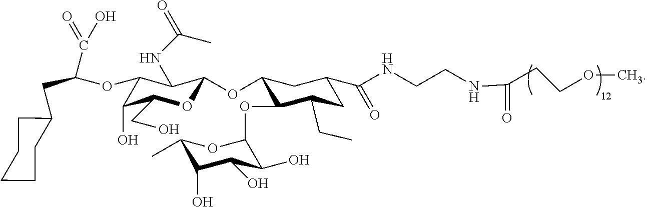

In some embodiments of a method disclosed herein, the at least one inhibitor is chosen from

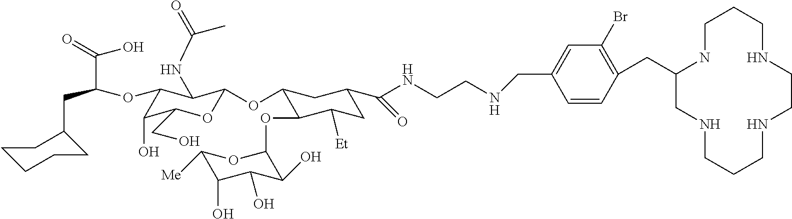 [SMILES: CCC1CC(C(=O)NCCNCc2ccc(CC3CNCCCNCCNCCCN3)c(Br)c2)CC(OC2OC(CO)C(O)C(O[C@@H](CC3CCCCC3)C(=O)O)C2NC(C)=O)C1OC1OC(C)C(O)C(O)C1O]

and pharmaceutically acceptable salts thereof.

In some embodiments of a method disclosed herein, the at least one inhibitor is

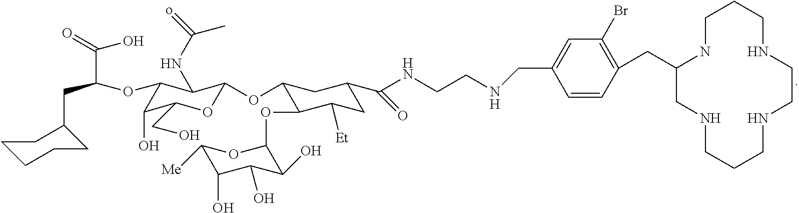

In some embodiments, the at least one FLT3 inhibitor is chosen from sorafenib, lestaurtinib, midostaurin, quizartinib, crenolanib, gilteritinib, and pharmaceutically acceptable salts of any of the foregoing. In some embodiments, the at least one FLT3 inhibitor is chosen from sorafenib, lestaurtinib, midostaurin, quizartinib, crenolanib, and gilteritinib.

In some embodiments of a method disclosed herein, the at least one FLT3 inhibitor is chosen from first generation multi-kinase inhibitors. In some embodiments, the at least one FLT3 inhibitor is chosen from sorafenib, lestaurtinib, midostaurin, and pharmaceutically acceptable salts of any of the foregoing.

In some embodiments of a method disclosed herein, the at least one FLT3 inhibitor is chosen from next generation inhibitors. In some embodiments, the at least one FLT3 inhibitor is chosen from quizartinib, crenolanib, gilteritinib, and pharmaceutically acceptable salts of any of the foregoing.

In some embodiments of a method disclosed herein, the at least one FLT3 inhibitor is chosen from sorafenib, quizartinib, and pharmaceutically acceptable salts of any of the foregoing. In some embodiments, the at least one FLT3 inhibitor is sorafenib. In some embodiments, the at least one FLT3 inhibitor is quizartinib.

In some embodiments of a method disclosed herein, the method comprises administering to the subject at least one FLT3 inhibitor and at least one E-selectin inhibitor.

In some embodiments of a method disclosed herein, the method comprises administering to the subject at least one FLT3 inhibitor and at least one CXCR4 inhibitor.

In some embodiments of a method disclosed herein, the method comprises administering to the subject at least one FLT3 inhibitor and at least one heterobifunctional inhibitor of E-selectin and CXCR4.

In some embodiments of a method disclosed herein, the method comprises administering to the subject at least one FLT3 inhibitor, at least one E-selectin inhibitor, and at least one CXCR4 inhibitor.

In some embodiments of a method disclosed herein, the method comprises administering a dose in the range of 5 mg/kg to 100 mg/kg (such as, e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one inhibitor.

In some embodiments of a method disclosed herein, the method comprises administering a dose in the range of 5 mg/kg to 100 mg/kg (such as, e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one E-selectin inhibitor.

In some embodiments of a method disclosed herein, the method comprises administering a dose in the range of 5 mg/kg to 100 mg/kg (such as, e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one CXCR4 inhibitor.

In some embodiments of a method disclosed herein, the method comprises administering a dose in the range of 5 mg/kg to 100 mg/kg (such as, e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one heterobifunctional inhibitor of E-selectin and CXCR4.

In some embodiments of a method disclosed herein, the method comprises administering a dose in the range of 0.5 mg/kg to 100 mg/kg (such as, e.g., 0.5 mg/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one FLT3 inhibitor.

In some embodiments of a method disclosed herein, the method comprises administering a fixed dose of 20 mg to 4000 mg (such as, e.g., 20 mg, 30 mg, 40 mg, 50 mg, 60 mg, 70 mg, 80 mg, 90 mg, 100 mg, 125 mg, 150 mg, 175 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, e.g., 800 mg to 3200 mg per day, 1000 mg to 2000 mg per day) per day of the at least one inhibitor.

In some embodiments of a method disclosed herein, the method comprises administering a fixed dose of 20 mg to 4000 mg (such as, e.g., 20 mg, 30 mg, 40 mg, 50 mg, 60 mg, 70 mg, 80 mg, 90 mg, 100 mg, 125 mg, 150 mg, 175 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, e.g., 800 mg to 3200 mg per day, 1000 mg to 2000 mg per day) per day of the at least one E-selectin inhibitor.

In some embodiments of a method disclosed herein, the method comprises administering a fixed dose of 20 mg to 4000 mg (such as, e.g., 20 mg, 30 mg, 40 mg, 50 mg, 60 mg, 70 mg, 80 mg, 90 mg, 100 mg, 125 mg, 150 mg, 175 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, e.g., 800 mg to 3200 mg per day, 1000 mg to 2000 mg per day) per day of the at least one CXCR4 inhibitor.

In some embodiments of a method disclosed herein, the method comprises administering a fixed dose of 20 mg to 4000 mg (such as, e.g., 20 mg, 30 mg, 40 mg, 50 mg, 60 mg, 70 mg, 80 mg, 90 mg, 100 mg, 125 mg, 150 mg, 175 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, e.g., 800 mg to 3200 mg per day, 1000 mg to 2000 mg per day) per day of the at least one heterobifunctional inhibitor of E-selectin and CXCR4.

In some embodiments of a method disclosed herein, the method comprises administering a fixed dose of 20 mg to 4000 mg (such as, e.g., 20 mg, 30 mg, 40 mg, 50 mg, 60 mg, 70 mg, 80 mg, 90 mg, 100 mg, 125 mg, 150 mg, 175 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, e.g., 800 mg to 3200 mg per day, 1000 mg to 2000 mg per day) per day of the at least one FLT3 inhibitor.

In some embodiments of a method disclosed herein, the method comprises administering a fixed dose of 20 mg to 4000 mg (such as, e.g., 20 mg, 30 mg, 40 mg, 50 mg, 60 mg, 70 mg, 80 mg, 90 mg, 100 mg, 125 mg, 150 mg, 175 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, e.g., 800 mg to 3200 mg per day, 1000 mg to 2000 mg per day) per day of sorafenib. In some embodiments, the method comprises administering a fixed dose of 800 mg per day of sorafenib. In some embodiments, the method comprises administering a fixed dose of 400 mg twice daily of sorafenib.

In some embodiments of a method disclosed herein, the method comprises administering a fixed dose of 20 mg to 500 mg (such as, e.g., 20 mg, 30 mg, 40 mg, 50 mg, 60 mg, 70 mg, 80 mg, 90 mg, 100 mg, 125 mg, 150 mg, 175 mg, 200 mg, 300 mg, 400 mg, 500 mg, e.g., 20 mg to 100 mg per day, 30 mg to 90 mg per day) per day of quizartinib. In some embodiments, the method comprises administering a fixed dose of 30 mg, 60 mg, or 90 mg per day of quizartinib. In some embodiments, the method comprises administering a fixed dose of 30 mg, 60 mg, or 90 mg once per day of quizartinib.

In some embodiments, the at least one E-selectin inhibitor is chosen from compounds of Formula (I):

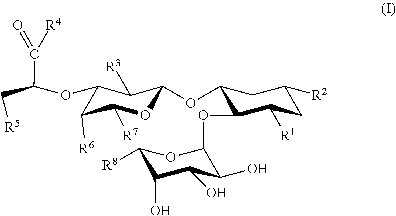

isomers of Formula (I), tautomers of Formula (I), and pharmaceutically acceptable salts of any of the foregoing, wherein:
R¹ is chosen from C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R² is chosen from H, —M, and -L-M;
R³ is chosen from —OH, —NH₂, —OC(═O)Y¹, —NHC(═O)Y¹, and —NHC(═O)NHY¹ groups, wherein Y¹ is chosen from C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₆₋₁₈ aryl, and C₁₋₁₃ heteroaryl groups;
R⁴ is chosen from —OH and —NZ¹Z² groups, wherein Z¹ and Z², which may be identical or different, are each independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Z¹ and Z² may together form a ring;
R⁵ is chosen from C₃₋₈ cycloalkyl groups;
R⁶ is chosen from —OH, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R⁷ is chosen from —CH₂OH, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups; R⁸ is chosen from C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
L is chosen from linker groups; and
M is a non-glycomimetic moiety chosen from polyethylene glycol, thiazolyl, chromenyl, —C(═O)NH(CH₂)₁₋₄NH₂, C₁₋₈ alkyl, and —C(═O)OY groups, wherein Y is chosen from C₁₋₄ alkyl, C₂₋₄ alkenyl, and C₂₋₄ alkynyl groups.

In some embodiments, the at least one E-selectin inhibitor is chosen from compounds of Formula (I), wherein the non-glycomimetic moiety comprises polyethylene glycol.

In some embodiments, the at least one E-selectin inhibitor is chosen from compounds of Formula (I), wherein L is —C(═O)NH(CH₂)₁₋₄NHC(═O)— and the non-glycomimetic moiety comprises polyethylene glycol.

In some embodiments, the at least one E-selectin inhibitor is chosen from compounds of Formula (Ia):

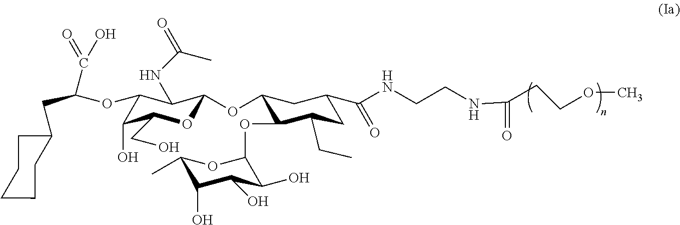

and pharmaceutically acceptable salts thereof, wherein n is chosen from integers ranging from 1 to 100. In some embodiments, n is chosen from 4, 8, 12, 16, 20, 24, and 28. In some embodiments n is 12.

In some embodiments, the at least one E-selectin inhibitor is chosen from Compound B:

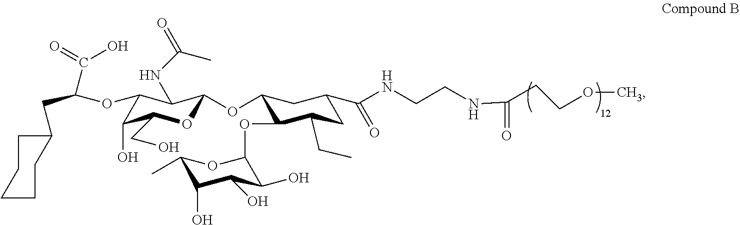

and pharmaceutically acceptable salts thereof.

In some embodiments, the at least one heterobifunctional inhibitor of E-selectin and CXCR4 is chosen from compounds of Formula (II):

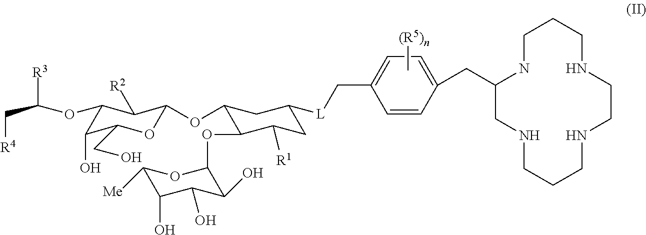

isomers of Formula (H), tautomers of Formula (H), and pharmaceutically acceptable salts of any of the foregoing, wherein:
R¹ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R² is chosen from —OH, —NH₂, —OC(═O)Y¹, —NHC(═O)Y¹, and —NHC(═O)NHY¹ groups, wherein Y¹ is chosen from C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₆₋₁₈ aryl, and C₁₋₁₃ heteroaryl groups;
R³ is chosen from —CN, —CH₂CN, and —C(═O)Y² groups, wherein Y² is chosen from C₁₋₈ alkyl, C₂₋₈ alkenyl, C₂₋₈ alkynyl, —OZ¹, —NHOH, —NHOCH₃, —NHCN, and —NZ¹Z² groups, wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Z¹ and Z² may together form a ring;
R⁴ is chosen from C₃₋₈ cycloalkyl groups;
R⁵ is independently chosen from H, halo, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
n is chosen from integers ranging from 1 to 4; and
L is chosen from linker groups.

In some embodiments, the at least one heterobifunctional inhibitor of E-selectin and CXCR4 is chosen from compounds of Formula (IIa):

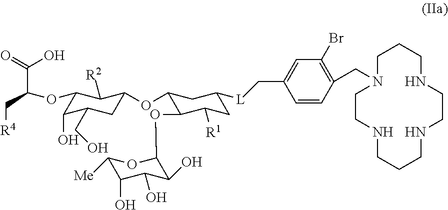

and pharmaceutically acceptable salts thereof.

In some embodiments, the at least one heterobifunctional inhibitor of E-selectin and CXCR4 is chosen from Compound A:

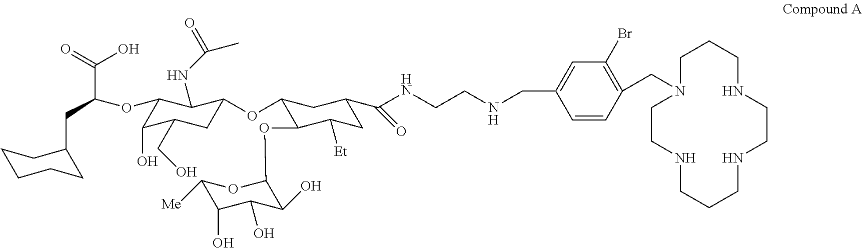 [SMILES: CCC1CC(C(=O)NCCNCc2ccc(CN3CCCNCCNCCCNCC3)c(Br)c2)CC(OC2CC(CO)C(O)C(O[C@@H](CC3CCCCC3)C(=O)O)C2NC(C)=O)C1OC1OC(C)C(O)C(O)C1O]

and pharmaceutically acceptable salts thereof.

In some embodiments, the at least one E-selectin inhibitor is a heterobifunctional pan-selectin antagonist chosen from compounds of Formula (III):

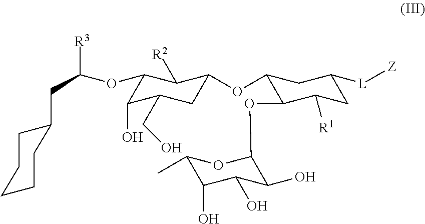

isomers of Formula (III), tautomers of Formula (III), and pharmaceutically acceptable salts of any of the foregoing, wherein:
R¹ is chosen from H, C₁₋₈ alkyl, C₂₋₈ alkenyl, C₂₋₈ alkynyl, C₄₋₁₆ cycloalkylalkyl

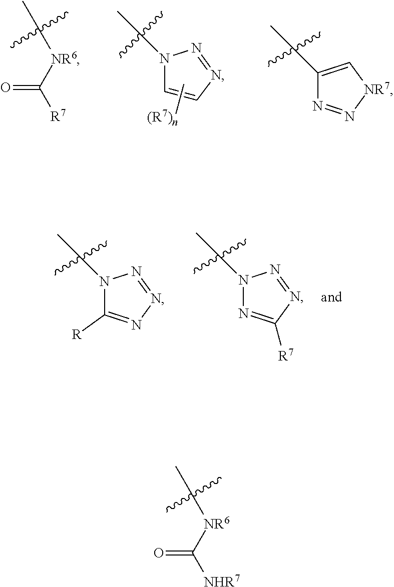

groups,
R² is chosen from C₁₋₈ alkyl, C₂₋₈ alkenyl, C₂₋₈ alkynyl, C₄₋₁₆ cycloalkylalkyl, —OH, —OX¹, halo, —NH₂, —OC(═O)X¹, —NHC(═O)X¹, and —NHC(═O)NHX¹ groups, wherein X¹ is chosen from C₁₋₈ alkyl, C₂₋₈ alkenyl, C₂₋₈ alkynyl, C₄₋₁₆cycloalkylalkyl, C₂₋₁₂ heterocyclyl, C₆₋₁₈ aryl, and C₁₋₁₃ heteroaryl groups;
R³ is chosen from —CN, —CH₂CN, and —C(═O)X² groups, wherein X² is chosen from C₁₋₈ alkyl, C₂₋₈ alkenyl, C₂₋₈ alkynyl, —OY², —NHOH, —NHOCH₃, —NHCN, and —NY²Y³ groups, wherein Y² and Y³, which may be identical or different, are independently chosen from H, C₁₋₈ alkyl, C₂₋₈ alkenyl, C₂₋₈ alkynyl, and C₄₋₁₆ cycloalkylalkyl groups, wherein Y² and Y³ may join together to form a ring,
R⁶ is chosen from H, C₁₋₈ alkyl, C₂₋₈ alkenyl, C₂₋₈ alkynyl, C₄₋₁₆ cycloalkylalkyl, and —C(═)R⁷ groups;
each R⁷ is independently chosen from H, C₁₋₈ alkyl, C₂₋₈ alkenyl, C₂₋₈ alkynyl, C₄₋₁₆ cycloalkylalkyl,

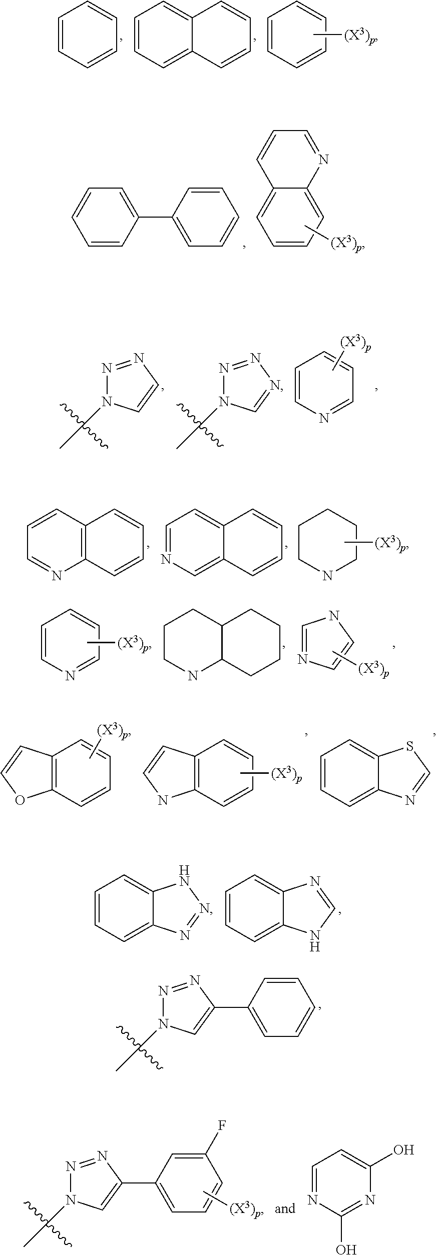

groups, wherein each X³ is independently chosen from H, —OH, Cl, F, N₃, —NH₂, C₁₋₈ alkyl, C₂₋₈ alkenyl, C₂₋₈ alkynyl, C₆₋₁₄ aryl, —OC₁₋₈ alkyl, —OC₂₋₈ alkenyl, —OC₂₋₈ alkynyl, and —OC₆₋₁₄ aryl groups, wherein any of the above ring compounds may be substituted with one to three groups independently chosen from Cl, F, C₁₋₈ alkyl, C₂₋₈ alkenyl, C₂₋₈ alkynyl, C₆₋₁₄ aryl, and —OY⁴ groups, wherein Y⁴ is chosen from H, C₁₋₈ alkyl, C₂₋₈ alkenyl, C₂₋₈ alkynyl, and C₆₋₁₄ aryl groups;
n is chosen from integers ranging from 0 to 2;
p is chosen from integers ranging from 0 to 3;
L is chosen from linker groups; and
Z is chosen from benzyl amino sulfonic acid groups.

Benzyl amino sulfonic acids (BASAs) are low molecular weight sulfated compounds which have the ability to interact with a selectin. The interaction modulates or assists in the modulation (e.g., inhibition or enhancement) of a selectin-mediated function (e.g., an intercellular interaction). They exist as either their protonated acid form, or as a sodium salt, although sodium may be replaced with potassium or any other pharmaceutically acceptable counterion.

Further disclosure regarding BASAs suitable for the disclosed compounds may be found in U.S. Reissue Patent No. RE44,778, issued Feb. 25, 2014, and U.S. Publication No. US2018/0369205, published Dec. 27, 2018, which are hereby incorporated by reference.

In some embodiments, the at least one E-selectin inhibitor is a heterobifunctional pan-selectin antagonist chosen from compounds of Formula (IIIa):

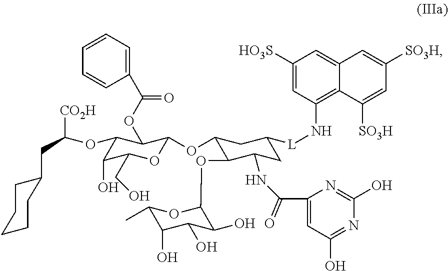

tautomers of Formula (IIIa), and pharmaceutically acceptable salts of any of the foregoing.

In some embodiments, the at least one E-selectin inhibitor is a heterobifunctional pan-selectin antagonist chosen from Compound C:

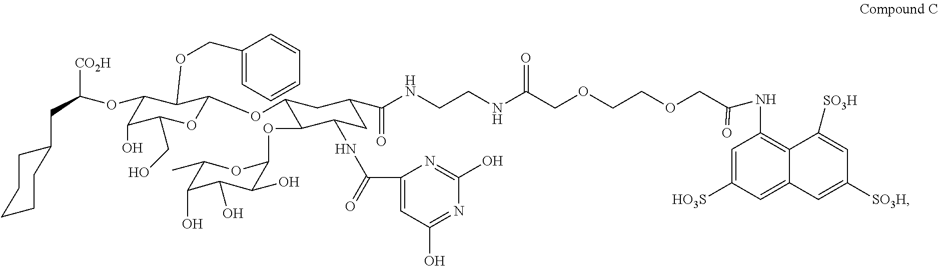 [SMILES: CC1OC(OC2C(NC(=O)c3cc(O)nc(O)n3)CC(C(=O)NCCNC(=O)COCCOCC(=O)Nc3cc(S(=O)(=O)O)cc4cc(S(=O)(=O)O)cc(S(=O)(=O)O)c34)CC2OC2OC(CO)C(O)C(O[C@@H](CC3CCCCC3)C(=O)O)C2OCc2ccccc2)C(O)C(O)C1O]

tautomers of Compound C, and pharmaceutically acceptable salts of any of the foregoing.

In some embodiments, the linker groups of Formula (I), Formula (II), and/or Formula (III) are independently chosen from groups comprising spacer groups, such spacer groups as, for example, —(CH₂)ₚ— and —O(CH₂)p—, wherein p is chosen from integers ranging from 1 to 30. In some embodiments, p is chosen from integers ranging from 1 to 20.

Other non-limiting examples of spacer groups include carbonyl groups and carbonyl-containing groups such as, for example, amide groups.

In some embodiments, the linker group of Formula (I), Formula (II), and/or Formula (III) is chosen from

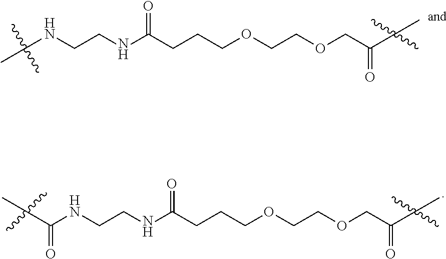 [SMILES: *NCCNC(=O)CCCOCCOCC(*)=O ; *C(=O)COCCOCCCC(=O)NCCNC(*)=O]

In some embodiments, the linker group of Formula (I), Formula (II), and/or Formula (III) is chosen from

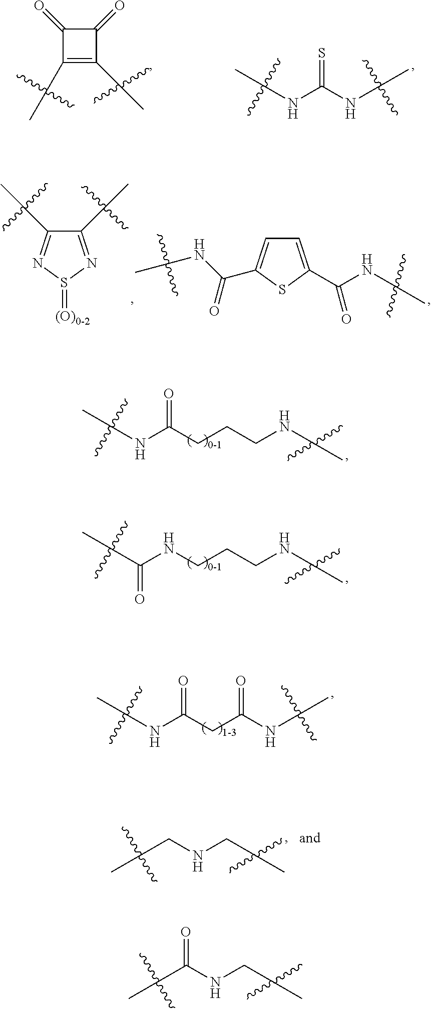

Other linker groups, such as, for example, polyethylene glycols (PEGs) and —C(═O)—NH—(CH₂)ₚ—C(═O)—NH—, wherein p is chosen from integers ranging from 1 to 30, or wherein p is chosen from integers ranging from 1 to 20, will be familiar to those of ordinary skill in the art and/or those in possession of the present disclosure.

In some embodiments, the linker group of Formula (I), Formula (II), and/or Formula (III) is chosen from

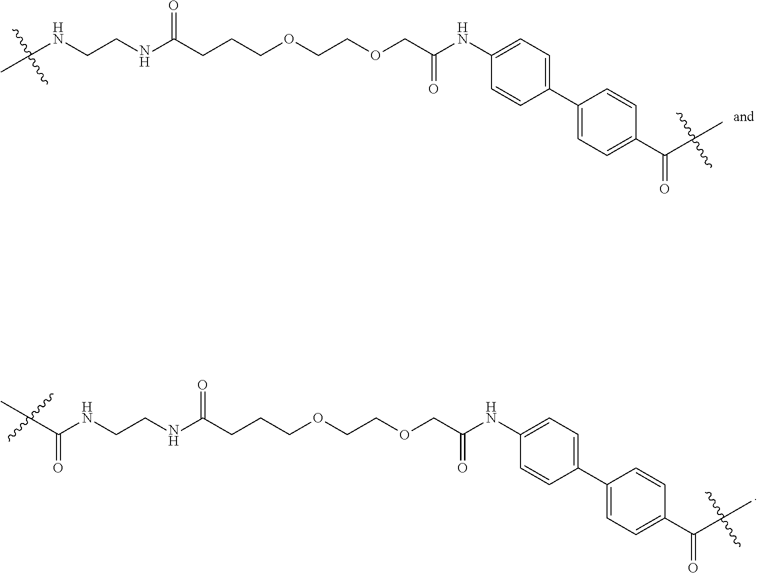 [SMILES: *NCCNC(=O)CCCOCCOCC(=O)Nc1ccc(-c2ccc(C(*)=O)cc2)cc1 ; *C(=O)NCCNC(=O)CCCOCCOCC(=O)Nc1ccc(-c2ccc(C(*)=O)cc2)cc1]

In some embodiments, the linker group of Formula (I), Formula (II), and/or Formula (III) is chosen from

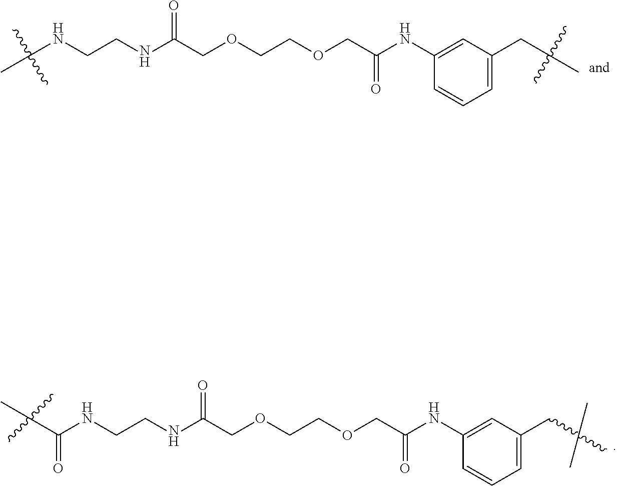

In some embodiments, the linker group of Formula (I), Formula (II), and/or Formula (III) is chosen from

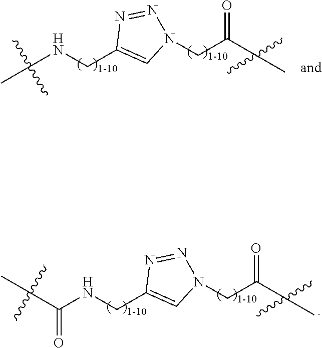

In some embodiments, the linker group of Formula (I), Formula (II), and/or Formula (III) is chosen from —C(═O)NH(CH₂)₂NH—, —CH₂NHCH₂—, and —C(═O)NHCH₂—. In some embodiments, the linker group is —C(═O)NH(CH₂)₂NH—.

In some embodiments, the at least one E-selectin inhibitor is chosen from compounds of Formula (IV):

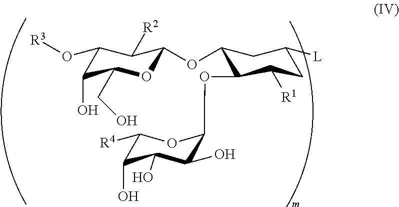

prodrugs of Formula (IV), isomers of Formula (IV), tautomers of Formula (IV), and pharmaceutically acceptable salts of any of the foregoing, wherein
each R¹, which may be identical or different, is independently chosen from H, C₁₋₁₂ alkyl, C₂₋₁₂ alkenyl, C₂₋₁₂ alkynyl, and —NHC(═O)R⁵ groups, wherein each R, which may be identical or different, is independently chosen from C₁₋₁₂ alkyl, C₂₋₁₂ alkenyl, C₂₋₁₂ alkynyl, C₆₋₁₈ aryl, and C₁₋₁₃ heteroaryl groups;
each R², which may be identical or different, is independently chosen from halo, —OY¹, —NY¹Y², —OC(═O)Y¹, —NHC(═O)Y¹, and —NHC(═O)NY¹Y² groups, wherein each Y¹ and each Y², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₆₋₁₈ aryl, and C₁₋₁₃ heteroaryl groups, wherein Y¹ and Y² may join together along with the nitrogen atom to which they are attached to form a ring;
each R³, which may be identical or different, is independently chosen from

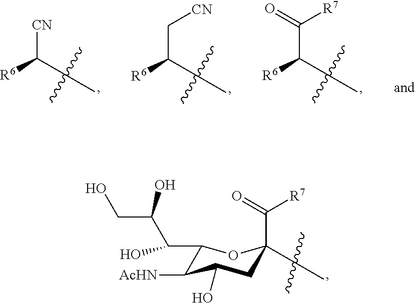

wherein each R⁶, which may be identical or different, is independently chosen from H, C₁₋₁₂ alkyl and C₁₋₁₂ haloalkyl groups, and wherein each R⁷, which may be identical or different, is independently chosen from C₁₋₈ alkyl, C₂₋₈ alkenyl, C₂₋₈ alkynyl, —OY³, —NHOH, —NHOCH₃, —NHCN, and —NY³Y⁴ groups, wherein each Y³ and each Y⁴, which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Y³ and Y⁴ may join together along with the nitrogen atom to which they are attached to form a ring;
each R⁴, which may be identical or different, is independently chosen from —CN, C₁₋₄ alkyl, and C₁₋₄ haloalkyl groups;
m is chosen from integers ranging from 2 to 256; and
L is chosen from linker groups.

In some embodiments, the at least one E-selectin inhibitor is chosen from compounds of Formula (V):

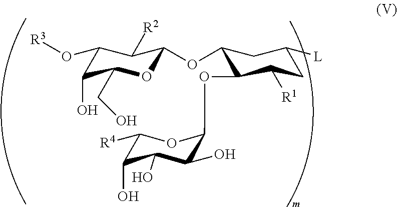

prodrugs of Formula (V), isomers of Formula (V), tautomers of Formula (V), and pharmaceutically acceptable salts of any of the foregoing, wherein
each R¹, which may be identical or different, is independently chosen from H, C₁₋₁₂ alkyl, C₂₋₁₂ alkenyl, C₂₋₁₂ alkynyl, and —NHC(═O)R⁵ groups, wherein each R⁵, which may be identical or different, is independently chosen from C₁₋₁₂ alkyl, C₂₋₁₂ alkenyl, C₂₋₁₂ alkynyl, C₆₋₁₈ aryl, and C₁₋₁₃ heteroaryl groups;
each R², which may be identical or different, is independently chosen from halo, —OY¹, —NY¹Y², —OC(═O)Y¹, —NHC(═O)Y¹, and —NHC(═O)NY¹Y² groups, wherein each Y¹ and each Y², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₆₋₁₈ aryl, and C₁₋₁₃ heteroaryl groups, wherein Y¹ and Y² may join together along with the nitrogen atom to which they are attached to form a ring;
each R³, which may be identical or different, is independently chosen from

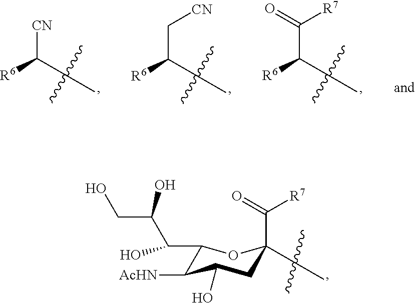

wherein each R⁶, which may be identical or different, is independently chosen from H, C₁₋₁₂ alkyl and C₁₋₁₂ haloalkyl groups, and wherein each R⁷, which may be identical or different, is independently chosen from C₁₋₈ alkyl, C₂₋₈ alkenyl, C₂₋₈ alkynyl, —OY³, —NHOH, —NHOCH₃, —NHCN, and —NY³Y⁴ groups, wherein each Y³ and each Y⁴, which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Y³ and Y⁴ may join together along with the nitrogen atom to which they are attached to form a ring;
each R⁴, which may be identical or different, is independently chosen from —CN, C₁₋₄ alkyl, and C₁₋₄ haloalkyl groups;
m is 2; and
L is chosen from

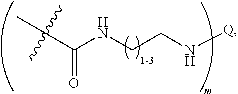

wherein Q is a chosen from

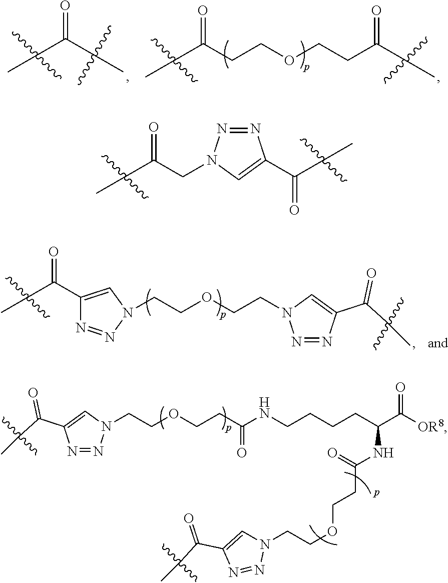

wherein R⁸ is chosen from H, C₁₋₈ alkyl, C₆₋₁₈ aryl, C₇₋₁₉ arylalkyl, and C₁₋₁₃ heteroaryl groups and each p, which may be identical or different, is independently chosen from integers ranging from 0 to 250.

In some embodiments, the at least one E-selectin inhibitor of Formula (IV) or Formula (V) is chosen from compounds of the following Formula (IVa/Va) (see definitions of L and m for Formula (IV) or (V) above):

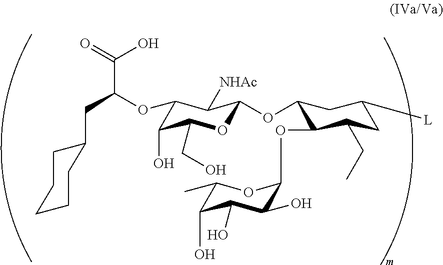

In some embodiments, the at least one E-selectin inhibitor of Formula (IV) or Formula (V) is chosen from compounds of the following Formula (IVb/Vb) (see definitions of L and m for Formula (IV) or (V) above):

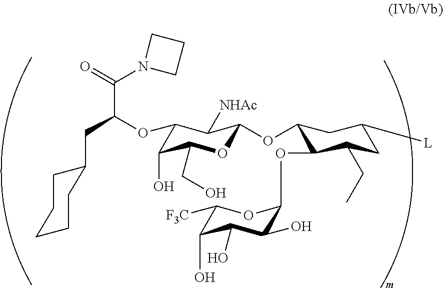

In some embodiments, the at least one E-selectin inhibitor is Compound D:

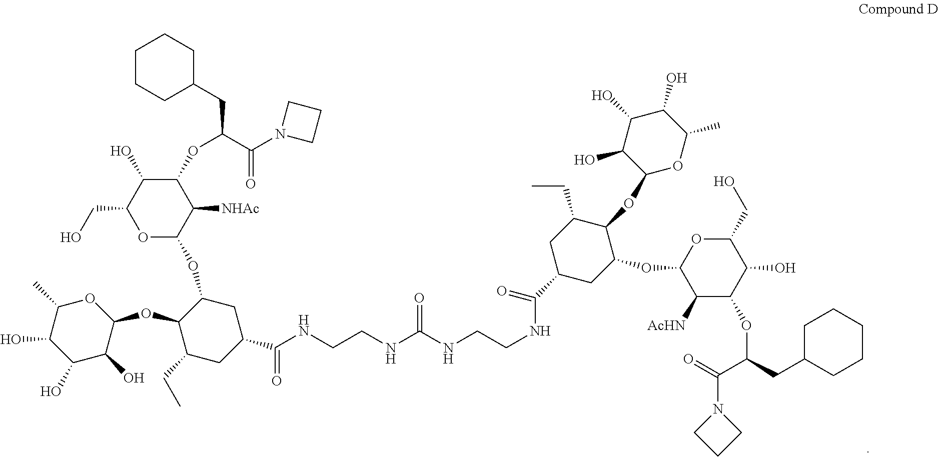 [SMILES: CC[C@H]1C[C@@H](C(=O)NCCNC(=O)NCCNC(=O)[C@@H]2C[C@H](CC)[C@@H](O[C@@H]3O[C@@H](C)[C@@H](O)[C@@H](O)[C@@H]3O)[C@H](O[C@@H]3O[C@H](CO)[C@H](O)[C@H](O[C@@H](CC4CCCCC4)C(=O)N4CCC4)[C@H]3NC(C)=O)C2)C[C@@H](O[C@@H]2O[C@H](CO)[C@H](O)[C@H](O[C@@H](CC3CCCCC3)C(=O)N3CCC3)[C@H]2NC(C)=O)[C@@H]1O[C@@H]1O[C@@H](C)[C@@H](O)[C@@H](O)[C@@H]1O]

In some embodiments, the at least one E-selectin inhibitor is a heterobifunctional inhibitor of E-selectin and Galectin-3, chosen from compounds of Formula (VI):

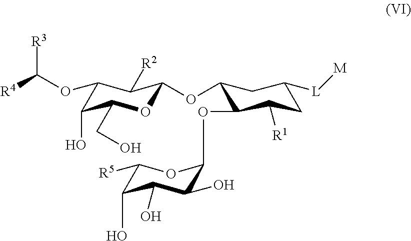

prodrugs of Formula (VI), isomers of Formula (VI), tautomers of Formula (VI), and pharmaceutically acceptable salts of any of the foregoing, wherein
R¹ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl,

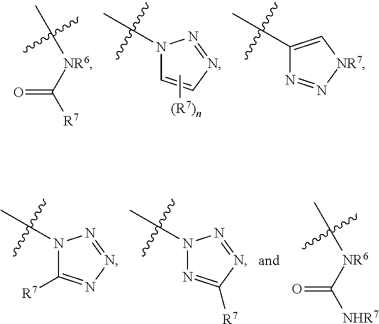

groups, wherein n is chosen from integers ranging from 0 to 2, R⁶ is chosen from H, C₁₋₈ alkyl, C₂₋₈ alkenyl, C₂₋₈ alkynyl, C₄₋₁₆ cycloalkylalkyl, and —C(═O)R⁷ groups, and each R⁷ is independently chosen from H, C₁₋₈ alkyl, C₂₋₈ alkenyl, C₂₋₈ alkynyl, C₄₋₁₆ cycloalkylalkyl, C₆₋₁₈ aryl, and C₁₋₁₃ heteroaryl groups;
R² is chosen from —OH, —OY¹, halo, —NH₂, —NY¹Y², —OC(═O)Y¹, —NHC(═O)Y¹, and —NHC(═O)NHY¹ groups, wherein Y¹ and Y², which may be the same or different, are independently chosen from C₁₋₈ alkyl, C₂₋₈ alkenyl, C₂₋₈ alkynyl, C₄₋₁₆ cycloalkylalkyl, C₂₋₁₂ heterocyclyl, C₆₋₁₈ aryl, and C₁₋₁₃ heteroaryl groups, wherein Y¹ and Y² may join together along with the nitrogen atom to which they are attached to form a ring;
R³ is chosen from —CN, —CH₂CN, and —C(═O)Y³ groups, wherein Y³ is chosen from C₁₋₈ alkyl, C₂₋₈ alkenyl, C₂₋₈ alkynyl, —OZ¹, —NHOH, —NHOCH₃, —NHCN, and —NZ¹Z² groups, wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and C₇₋₁₂ arylalkyl groups, wherein Z¹ and Z² may join together along with the nitrogen atom to which they are attached to form a ring;
R⁴ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, and C₆₋₁₈ aryl groups;
R⁵ is chosen from —CN, C₁₋₈ alkyl, and C₁₋₄ haloalkyl groups;
M is chosen from

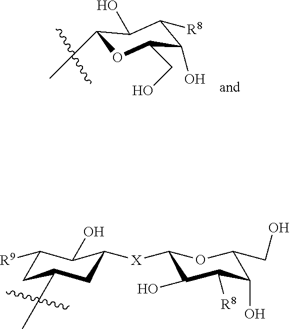

groups, wherein X is chosen from O and S, and R⁸ and R⁹, which may be identical or different, are independently chosen from C₆₋₁₈ aryl, C₁₋₁₃ heteroaryl, C₇₋₁₉ arylalkyl, C₇₋₁₉ arylalkoxy, C₂₋₁₄ heteroarylalkyl, C₂₋₁₄ heteroarylalkoxy, and —NHC(═O)Y⁴ groups, wherein Y⁴ is chosen from C₁₋₈ alkyl, C₂₋₁₂ heterocyclyl, C₆₋₁₈ aryl, and C₁₋₁₃ heteroaryl groups; and
L is chosen from linker groups.

In some embodiments, the at least one E-selectin inhibitor is chosen from compounds having the following Formulae:

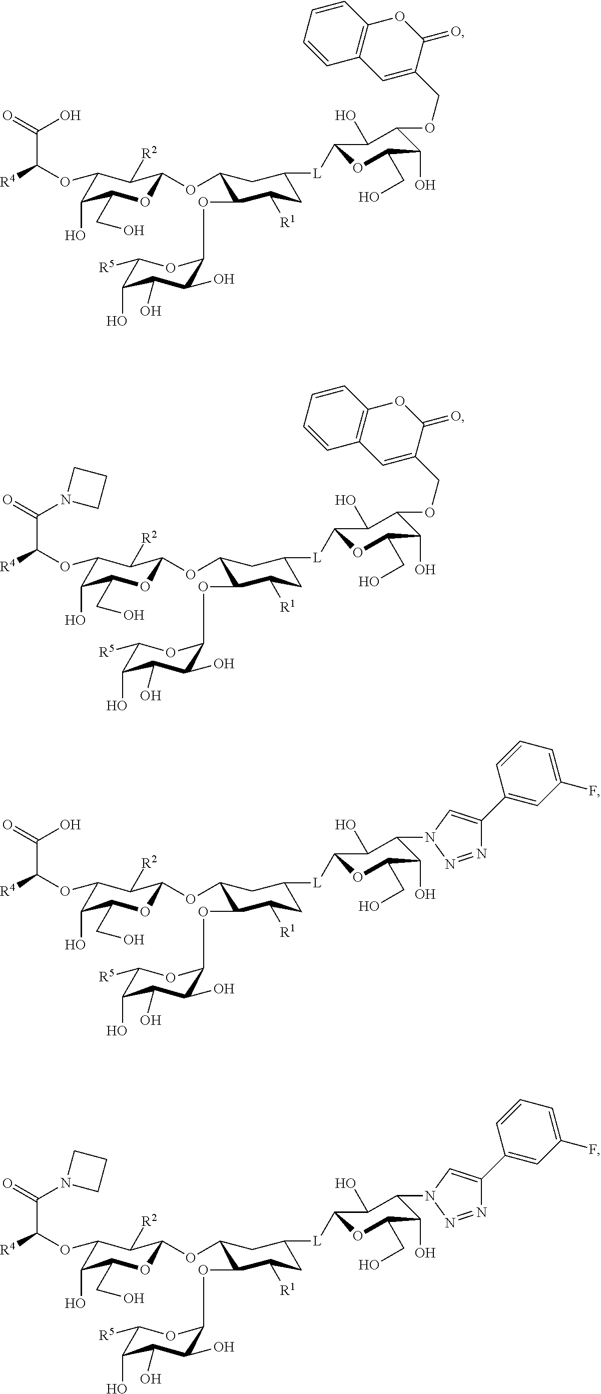

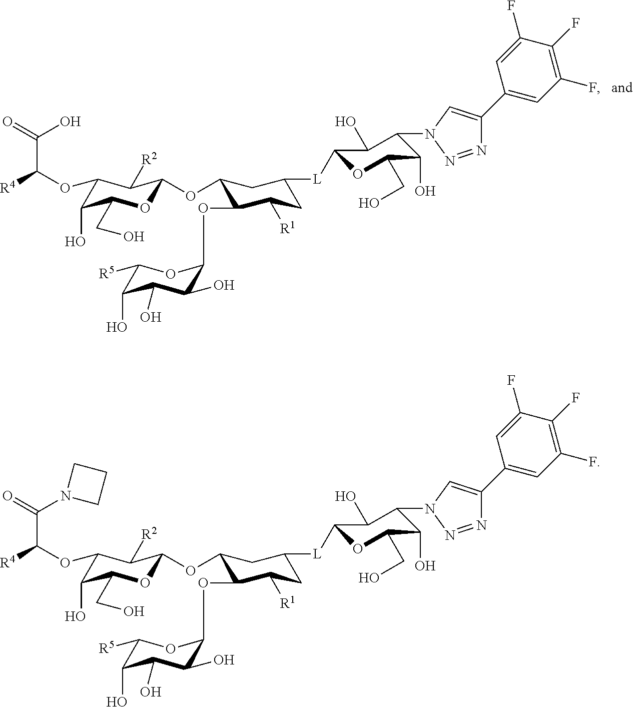

In some embodiments, the at least one E-selectin inhibitor is chosen from compounds having the following Formulae:

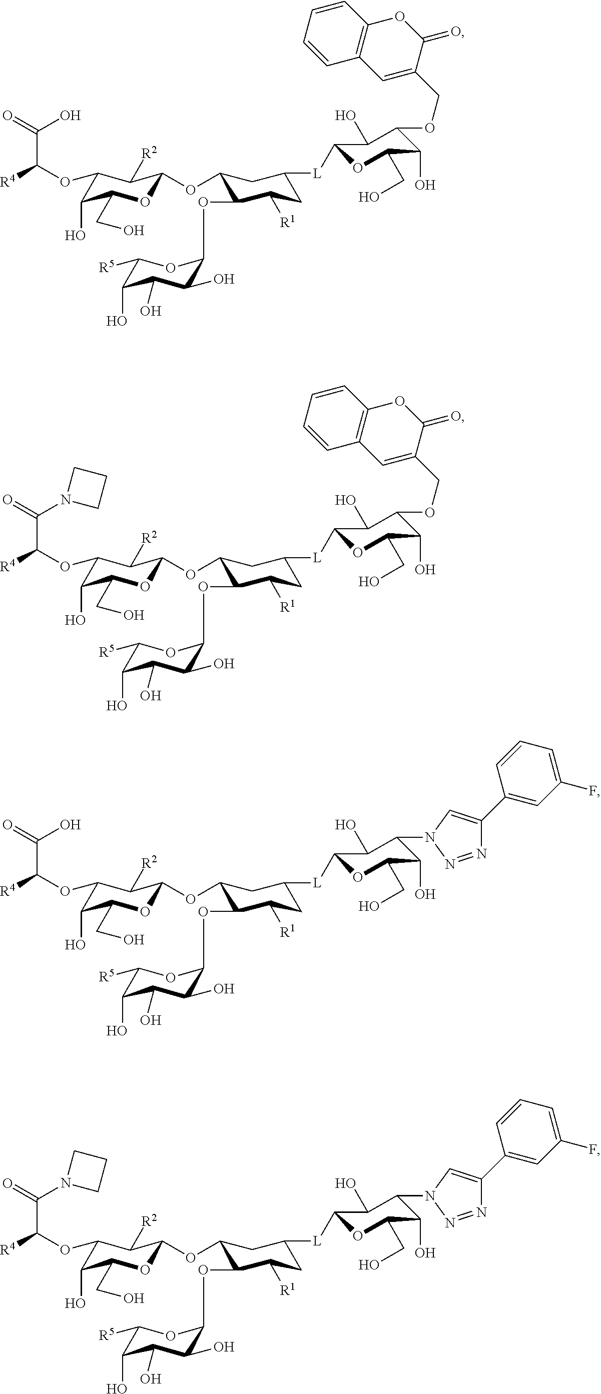

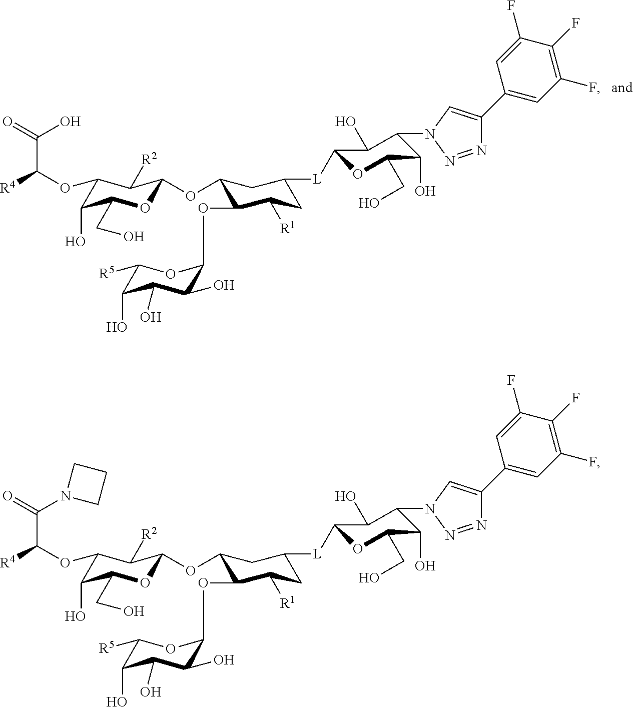

and pharmaceutically acceptable salts of any of the foregoing.

In some embodiments, the at least one E-selectin inhibitor is chosen from compounds having the following Formulae:

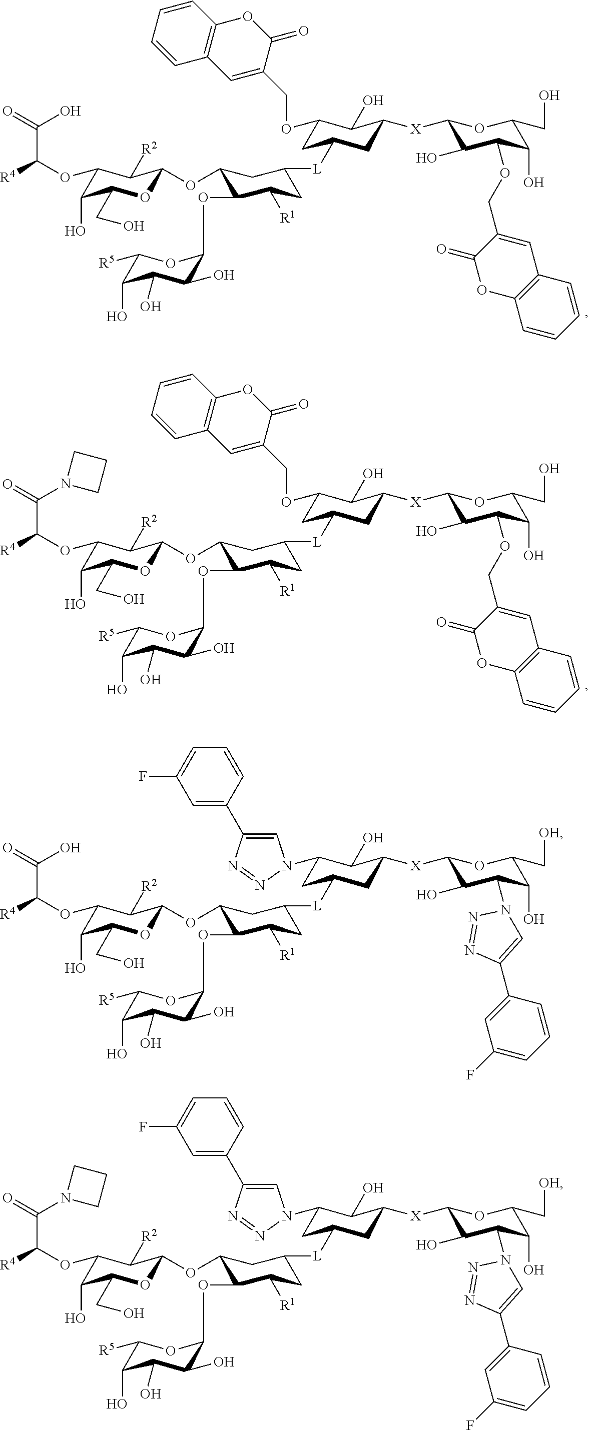

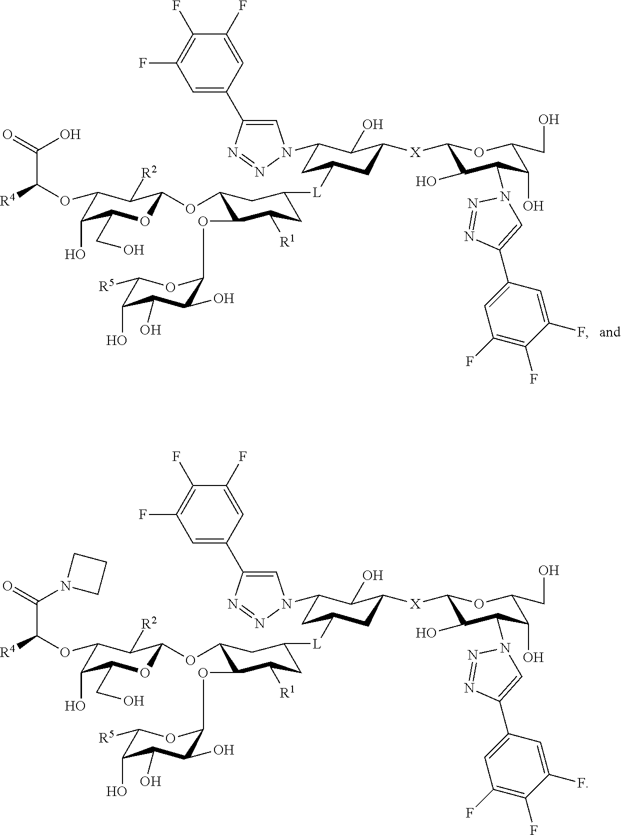

In some embodiments, the at least one E-selectin inhibitor is chosen from compounds having the following Formulae:

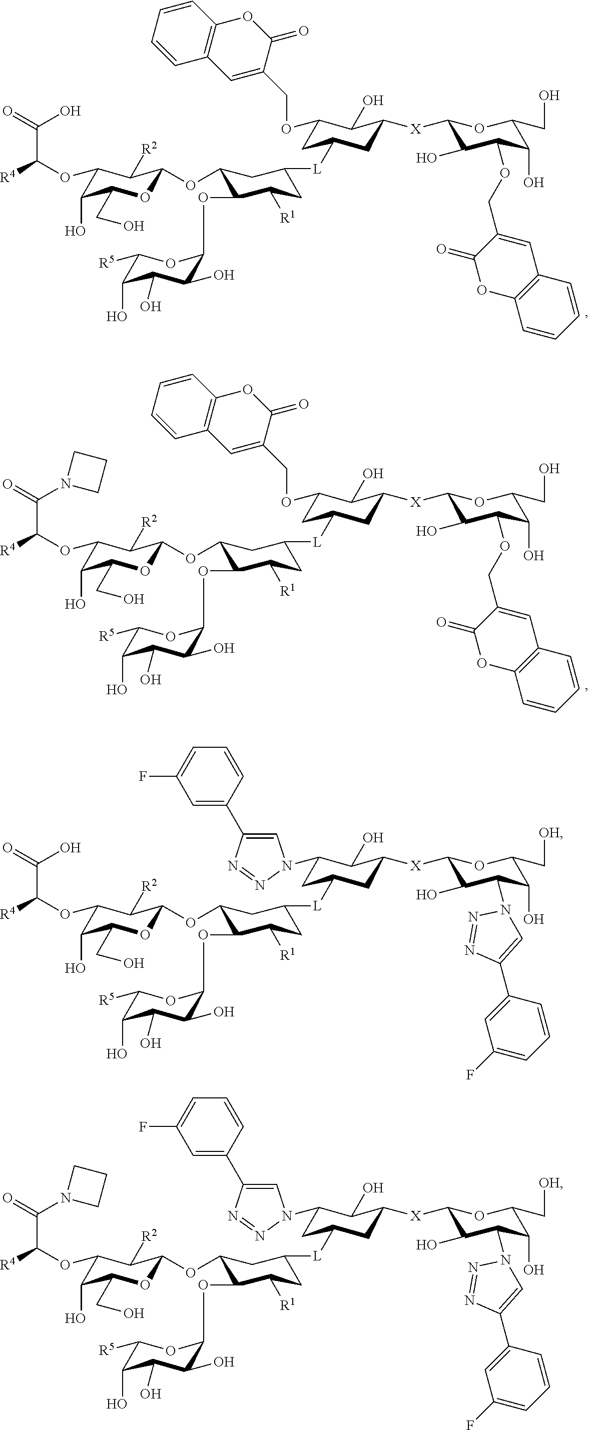

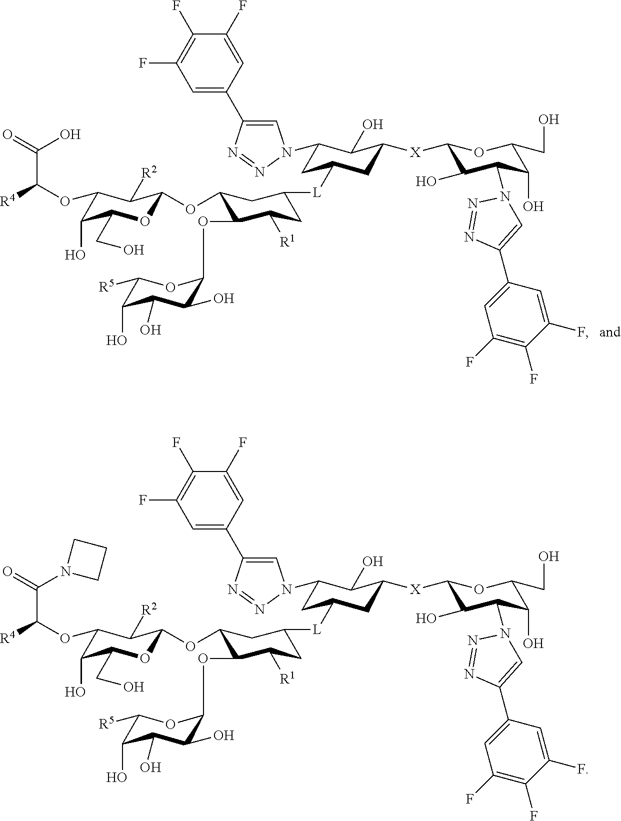

and pharmaceutically acceptable salts of any of the foregoing.

In some embodiments, the at least one E-selectin inhibitor is Compound E:

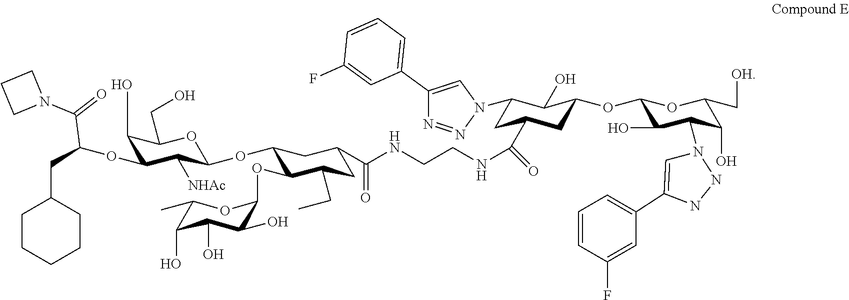

In some embodiments, the at least one E-selectin inhibitor is chosen from compounds of Formula (VII):

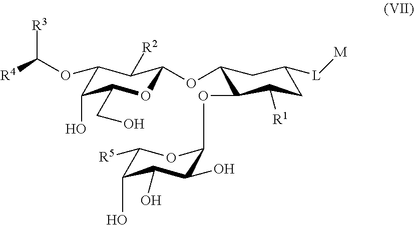

prodrugs of Formula (VII), isomers of Formula (VII), tautomers of Formula (VII), and pharmaceutically acceptable salts of any of the foregoing, wherein
R¹ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl,

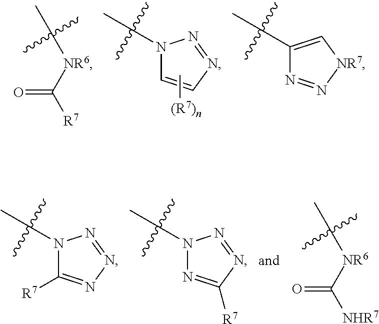

groups, wherein n is chosen from integers ranging from 0 to 2, R⁶ is chosen from H, C₁₋₈ alkyl, C₂₋₈ alkenyl, C₂₋₈ alkynyl, C₄₋₁₆ cycloalkylalkyl, and —C(═O)R⁷ groups, and each R⁷ is independently chosen from H, C₁₋₈ alkyl, C₂₋₈ alkenyl, C₂₋₈ alkynyl, C₄₋₁₆ cycloalkylalkyl, C₆₋₁₈ aryl, and C₁₋₁₃ heteroaryl groups;
R² is chosen from —OH, —OY¹, halo, —NH₂, —NY¹Y², —OC(═O)Y¹, —NHC(═O)Y¹, and —NHC(═O)NHY¹ groups, wherein Y¹ and Y², which may be the same or different, are independently chosen from C₁₋₈ alkyl, C₂₋₈ alkenyl, C₂₋₈ alkynyl, C₄₋₁₆ cycloalkylalkyl, C₂₋₁₂ heterocyclyl, C₆₋₁₈ aryl, and C₁₋₁₃ heteroaryl groups, or Y¹ and Y² join together along with the nitrogen atom to which they are attached to form a ring;
R³ is chosen from —CN, —CH₂CN, and —C(═O)Y³ groups, wherein Y³ is chosen from C₁₋₈ alkyl, C₂₋₈ alkenyl, C₂₋₈ alkynyl, —OZ¹, —NHOH, —NHOCH₃, —NHCN, and —NZ¹Z² groups, wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and C₇₋₁₂ arylalkyl groups, or Z¹ and Z² join together along with the nitrogen atom to which they are attached to form a ring;
R⁴ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, and C₆₋₁₈ aryl groups;
R⁵ is chosen from —CN, C₁₋₈ alkyl, and C₁₋₄ haloalkyl groups;
M is chosen from

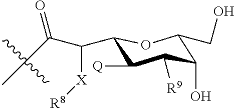

groups,
wherein
   X is chosen from —O—, —S—, —C—, and —N(R¹⁰)—, wherein R¹⁰ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups,
   Q is chosen from H, halo, and —OZ³ groups, wherein Z³ is chosen from H and C₁₋₈ alkyl groups,
R⁸ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, C₆₋₁₈ aryl, C₁₋₁₃ heteroaryl, C₇₋₁₉ arylalkyl, and C₂₋₁₄ heteroarylalkyl groups, wherein the C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, C₆₋₁₈ aryl, C₁₋₁₃ heteroaryl, C₇₋₁₉ arylalkyl, and C₂₋₁₄ heteroarylalkyl groups are optionally substituted with one or more groups independently chosen from halo, C₁₋₈ alkyl, C₁₋₈ hydroxyalkyl, C₁₋₈ haloalkyl, C₆₋₁₈ aryl, —OZ⁴, —C(═O)OZ⁴, —C(═O)NZ⁴Z⁵, and —SO₂Z⁴ groups, wherein Z⁴ and Z⁵, which may be identical or different, are independently chosen from H, C₁₋₈ alkyl, and C₁₋₈ haloalkyl groups, or Z⁴ and Z⁵ join together along with the nitrogen atom to which they are attached to form a ring,
R⁹ is chosen from C₆₋₁₈ aryl and C₁₋₁₃ heteroaryl groups, wherein the C₆₋₁₈ aryl and C₁₋₁₃ heteroaryl groups are optionally substituted with one or more groups independently chosen from R¹¹, C₁₋₈ alkyl, C₁₋₈ haloalkyl, —C(═O)OZ⁶, and —C(═O)NZ⁶Z⁷ groups, wherein R¹¹ is independently chosen from C₆₋₁₈ aryl groups optionally substituted with one or more groups independently chosen from halo, C₁₋₈ alkyl, —OZ⁸, —C(═O)OZ⁸, and —C(═O)NZ⁸Z⁹ groups, wherein Z⁶, Z⁷, Z⁸ and Z⁹, which may be identical or different, are independently chosen from H and C₁₋₈ alkyl groups, or Z⁶ and Z⁷ join together along with the nitrogen atom to which they are attached to form a ring and/or Z⁸ and Z⁹ join together along with the nitrogen atom to which they are attached to form a ring, and wherein each of Z³, Z⁴, Z⁵, Z⁶, Z⁷, Z⁸, and Z⁹ is optionally substituted with one or more groups independently chosen from halo and —OR¹² groups, wherein R¹² is independently chosen from H and C₁₋₈ alkyl groups; and
L is chosen from linker groups.

In some embodiments of Formula (VII), M is chosen from

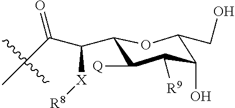

groups.

In some embodiments of Formula (VII), M is chosen from

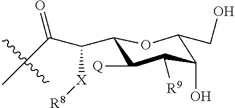

groups.

In some embodiments of Formula (VII), linker groups may be chosen from groups comprising spacer groups, such spacer groups as, for example, —(CH2)ₜ- and —O(CH2)ₜ-, wherein t is chosen from integers ranging from 1 to 20. Other non-limiting examples of spacer groups include carbonyl groups and carbonyl-containing groups such as, for example, amide groups. A non-limiting example of a spacer group is

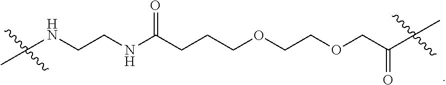 [SMILES: *NCCNC(=O)CCCOCCOCC(*)=O]

In some embodiments of Formula (VII), the linker group is chosen from

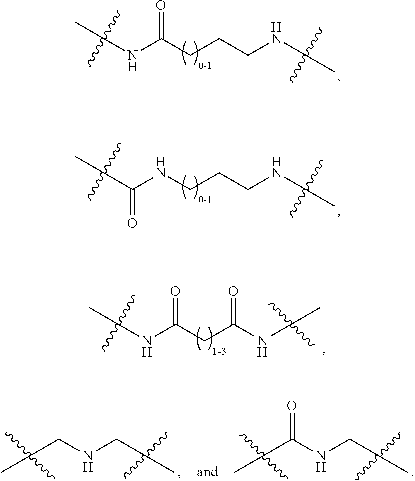

In some embodiments of Formula (VII), the linker group is chosen from polyethylene glycols (PEGs), —C(═O)NH(CH₂)ᵥO—, —C(═O)NH(CH₂)ᵥNHC(═O), —C(═O)NHC(═O)(CH₂)NH—, and —C(═O)NH(CH₂)ᵥC(═O)NH— groups, wherein v is chosen from integers ranging from 2 to 20. In some embodiments, v is chosen from integers ranging from 2 to 4. In some embodiments, v is 2. In some embodiments, v is 3. In some embodiments, v is 4.

In some embodiments of Formula (VII), the linker group is

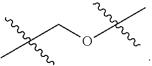 [SMILES: *CO*]

In some embodiments of Formula (VII), the linker group is

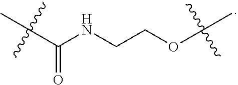 [SMILES: *OCCNC(*)=O]

In some embodiments of Formula (VII), the linker group is

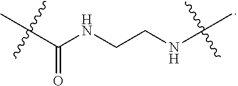 [SMILES: *NCCNC(*)=O]

In some embodiments of Formula (VII), the linker group is

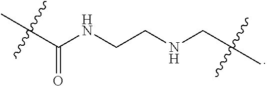 [SMILES: *CNCCNC(*)=O]

In some embodiments of Formula (VII), the linker group is

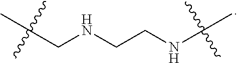 [SMILES: *CNCCN*]

In some embodiments of Formula (VII), the linker group is

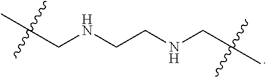 [SMILES: *CNCCNC*]

In some embodiments of Formula (VII), the linker group is

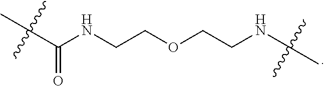 [SMILES: *NCCOCCNC(*)=O]

In some embodiments of Formula (VII), the linker group is

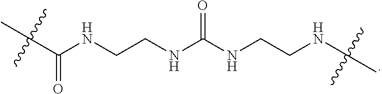 [SMILES: *NCCNC(=O)NCCNC(*)=O]

In some embodiments of Formula (VII), the linker group is

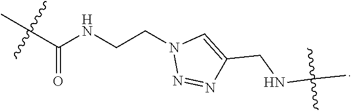 [SMILES: *NCc1cn(CCNC(*)=O)nn1]

Figures and examples illustrating the synthesis of compounds of Formula (VII) are shown in PCT International Application Publication No. WO 2020/139962, which is incorporated by reference herein in its entirety.

In some embodiments, the at least one E-selectin inhibitor is a multimeric inhibitor of E-selectin, Galectin-3, and/or CXCR4, chosen from compounds of Formula (VIII):

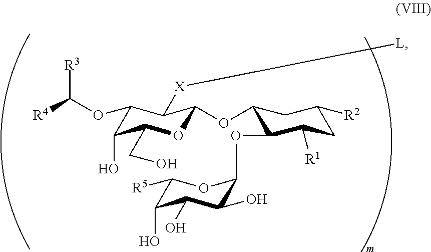

prodrugs of Formula (VIII), and pharmaceutically acceptable salts of any of the foregoing, wherein
each R¹, which may be identical or different, is independently chosen from H, C₁₋₁₂ alkyl, C₂₋₁₂ alkenyl, C₂₋₁₂ alkynyl, C₁₋₈ haloalkyl, C₂₋₈ haloalkenyl, C₂₋₈ haloalkynyl,

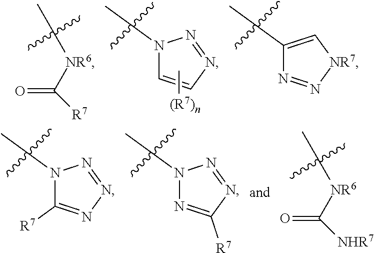

groups, wherein each n, which may be identical or different, is chosen from integers ranging from 0 to 2, each R⁶, which may be identical or different, is independently chosen from H, C₁₋₈ alkyl, C₂₋₈ alkenyl, C₂₋₈ alkynyl, C₄₋₁₆ cycloalkylalkyl, and —C(═O)R⁷ groups, and each R⁷, which may be identical or different, is independently chosen from H, C₁₋₈ alkyl, C₂₋₈ alkenyl, C₂₋₈ alkynyl, C₄₋₁₆ cycloalkylalkyl, C₆₋₁₈ aryl, and C₁₋₁₃ heteroaryl groups;
each R², which may be identical or different, is independently chosen from H, a non-glycomimetic moiety, and a linker-non-glycomimetic moiety, wherein each non-glycomimetic moiety, which may be identical or different, is independently chosen from galectin-3 inhibitors, CXCR4 chemokine receptor inhibitors, polyethylene glycol, thiazolyl, chromenyl, C₁₋₈ alkyl, R⁸, C₆₋₁₈ aryl-R⁸, C₁₋₁₂ heteroaryl-R⁸,

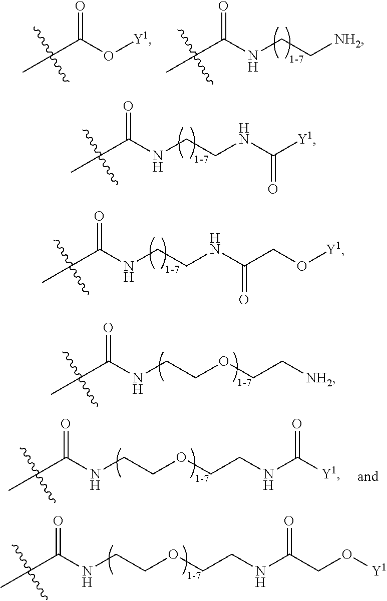

groups,
wherein each Y¹, which may be identical or different, is independently chosen from C₁₋₄ alkyl, C₂₋₄ alkenyl, and C₂₋₄ alkynyl groups and wherein each R⁸, which may be identical or different, is independently chosen from C₁₋₁₂ alkyl groups substituted with at least one substituent chosen from —OH, —OSO₃Q, —OPO₃Q₂, —CO₂Q, and —SO₃Q groups and C₂₋₁₂ alkenyl groups substituted with at least one substituent chosen from —OH, —OSO₃Q, —OPO₃Q₂, —CO₂Q, and —SO₃Q groups, wherein each Q, which may be identical or different, is independently chosen from H and pharmaceutically acceptable cations;
each R³, which may be identical or different, is independently chosen from —CN, —CH₂CN, and —C(═O)Y² groups, wherein each Y², which may be identical or different, is independently chosen from C₁₋₈ alkyl, C₂₋₈ alkenyl, C₂₋₈ alkynyl, —OZ¹, —NHOH, —NHOCH₃, —NHCN, and —NZ¹Z² groups, wherein each Z¹ and Z², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, and C₇₋₁₂ arylalkyl groups, wherein Z¹ and Z² may join together along with the nitrogen atom to which they are attached to form a ring;
each R⁴, which may be identical or different, is independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₄₋₁₆ cycloalkylalkyl, and C₆₋₁₈ aryl groups;
each R⁵, which may be identical or different, is independently chosen from —CN, C₁₋₁₂ alkyl, and C₁₋₁₂ haloalkyl groups;
each X, which may be identical or different, is independently chosen from —O— and —N(R⁹)—, wherein each R⁹, which may be identical or different, is independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
m is chosen from integers ranging from 2 to 256; and
L is independently chosen from linker groups.

In some embodiments of Formula (VIII), at least one linker groups is chosen from groups comprising spacer groups, such spacer groups as, for example, —(CH2)z- and —O(CH2)z-, wherein z is chosen from integers ranging from 1 to 250. Other non-limiting examples of spacer groups include carbonyl groups and carbonyl-containing groups such as, for example, amide groups. A non-limiting example of a spacer group is

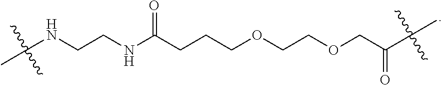 [SMILES: *NCCNC(=O)CCCOCCOCC(*)=O]

In some embodiments of Formula (VIII), at least one linker group is chosen from

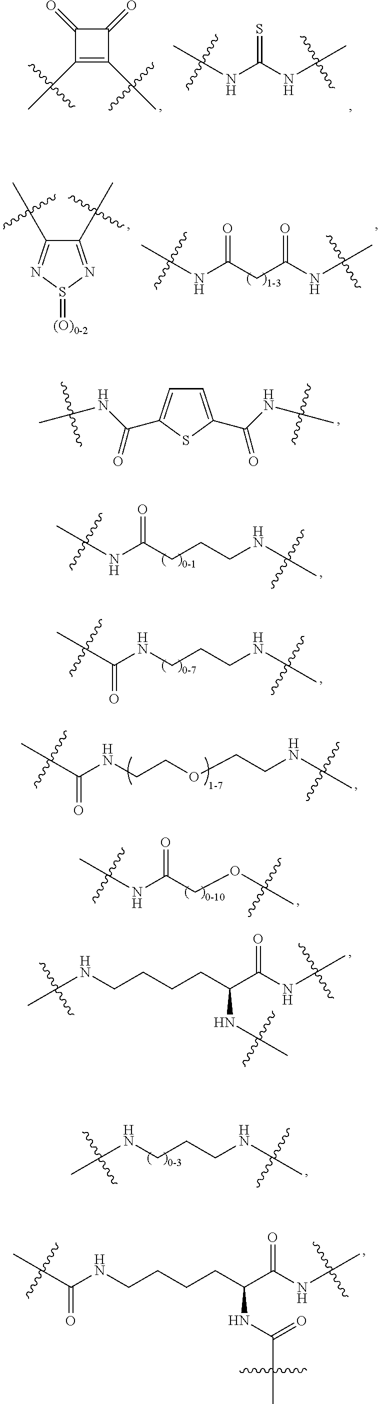

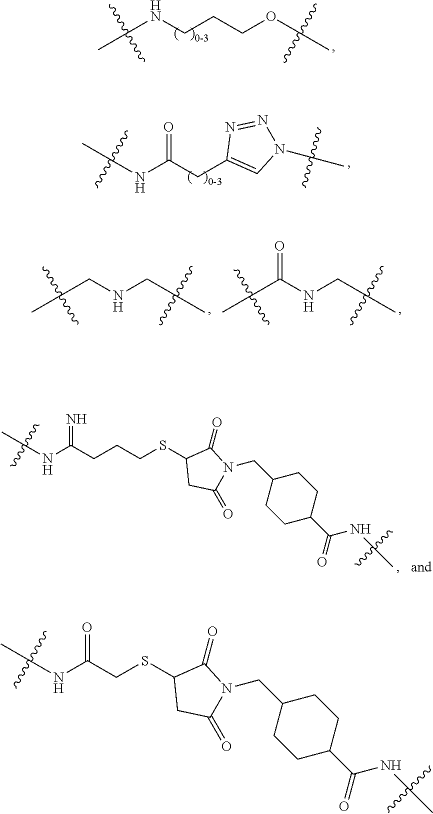

groups.

Other linker groups for certain embodiments of Formula (VIII), such as, for example, polyethylene glycols (PEGs) and —C(═O)—NH—(CH₂)_{z}—C(═O)—NH—, wherein z is chosen from integers ranging from 1 to 250, will be familiar to those of ordinary skill in the art and/or those in possession of the present disclosure.

In some embodiments of Formula (VIII), at least one linker group is

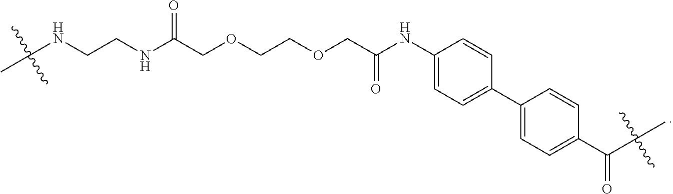 [SMILES: *NCCNC(=O)COCCOCC(=O)Nc1ccc(-c2ccc(C(*)=O)cc2)cc1]

In some embodiments of Formula (VIII), at least one linker group is

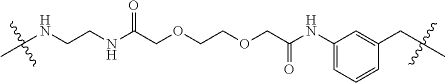 [SMILES: *Cc1cccc(NC(=O)COCCOCC(=O)NCCN*)c1]

In some embodiments of Formula (VIII), at least one linker group is chosen from —C(═O)NH(CH₂)₂NH—, —CH₂NHCH₂—, and —C(═O)NHCH₂—. In some embodiments of Formula (VIII), at least one linker group is —C(═O)NH(CH₂)₂NH—.

In some embodiments of Formula (VIII), L is chosen from dendrimers. In some embodiments of Formula (VIII), L is chosen from polyamidoamine (“PAMAM”) dendrimers. In some embodiments of Formula (VIII), L is chosen from PAMAM dendrimers comprising succinamic acid. In some embodiments of Formula (VIII), L is PAMAM GO generating a tetramer. In some embodiments of Formula (VIII), L is PAMAM G1 generating an octamer. In some embodiments of Formula (VIII), L is PAMAM G2 generating a 16-mer. In some embodiments of Formula (VIII), L is PAMAM G3 generating a 32-mer. In some embodiments of Formula (VIII), L is PAMAM G4 generating a 64-mer. In some embodiments, L is PAMAM G5 generating a 128-mer.

In some embodiments of Formula (VIII), m is 2 and L is chosen from

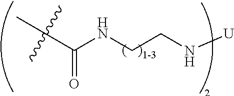

groups,
wherein U is chosen from

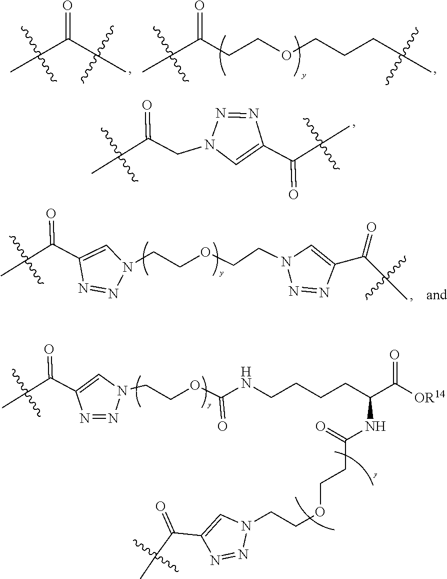

groups,
wherein R¹⁴ is chosen from H, C₁₋₈ alkyl, C₆₋₁₈ aryl, C₇₋₁₉ arylalkyl, and C₁₋₁₃ heteroaryl groups and each y, which may be identical or different, is independently chosen from integers ranging from 0 to 250. In some embodiments of Formula (VIII), R¹⁴ is chosen from C₁₋₈ alkyl. In some embodiments of Formula (VIII), R¹⁴ is chosen from C₇₋₁₉ arylalkyl. In some embodiments of Formula (VIII), R¹⁴ is H. In some embodiments of Formula (VIII), R¹⁴ is benzyl.

In some embodiments of Formula (VIII), L is chosen from

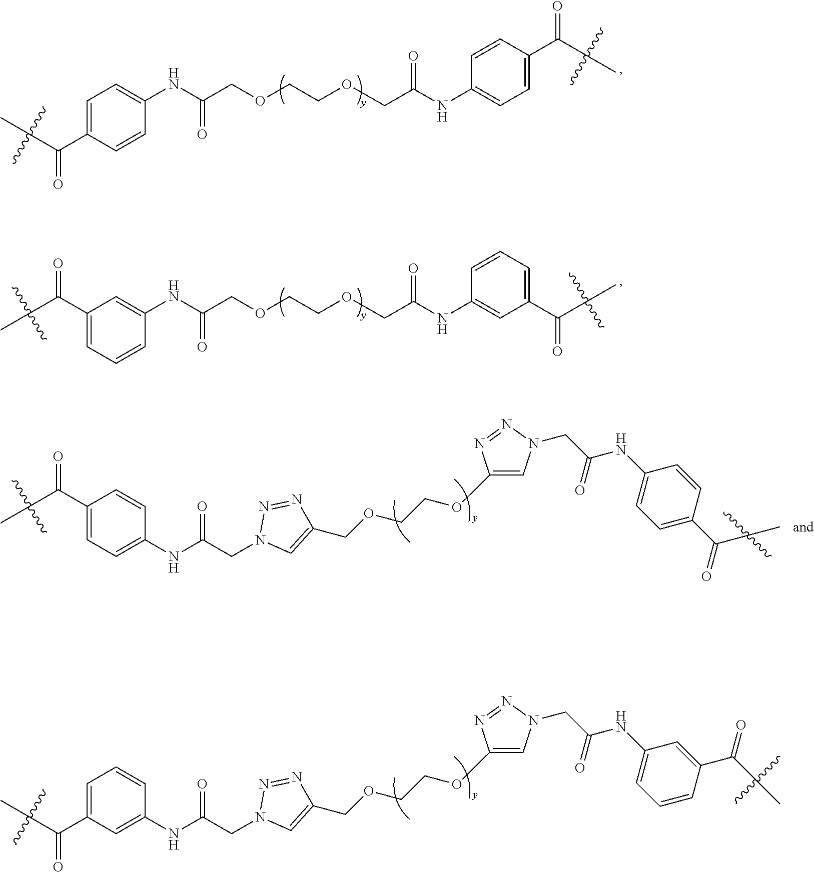

wherein y is chosen from integers ranging from 0 to 250.

In some embodiments of Formula (VIII), L is chosen from

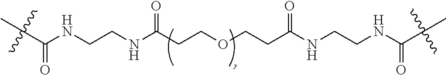

groups,
wherein y is chosen from integers ranging from 0 to 250.

In some embodiments of Formula (VIII), L is

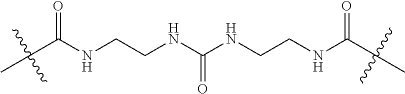 [SMILES: *C(=O)NCCNC(=O)NCCNC(*)=O]

In some embodiments of Formula (VIII), L is chosen from

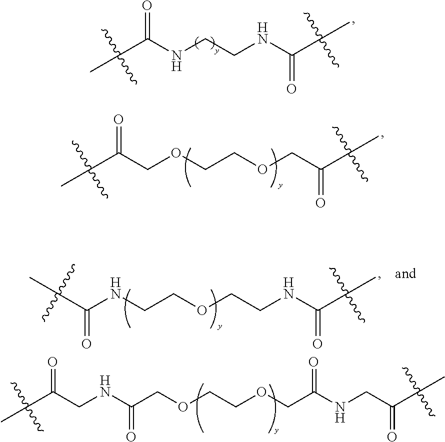

groups,
wherein y is chosen from integers ranging from 0 to 250.

In some embodiments of Formula (VIII), L is chosen from

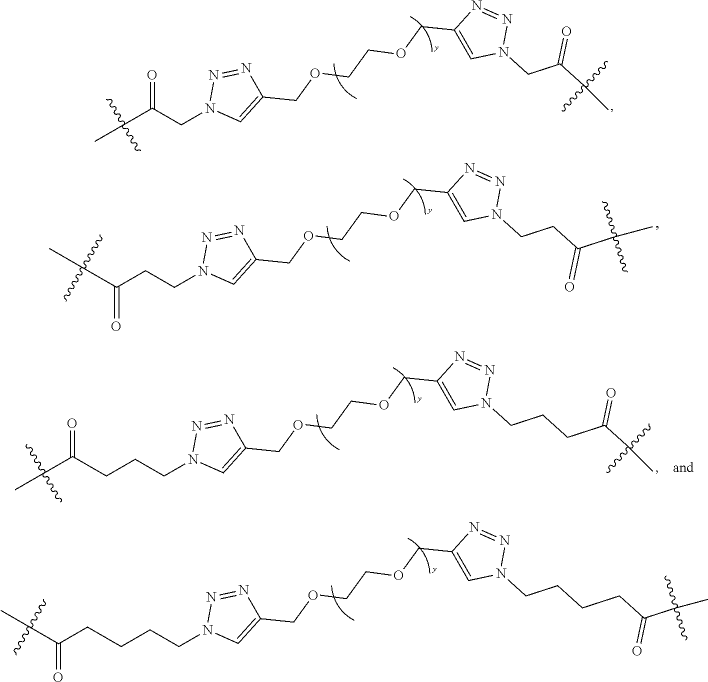

groups,
wherein y is chosen from integers ranging from 0 to 250.

In some embodiments of Formula (VIII), L is chosen from

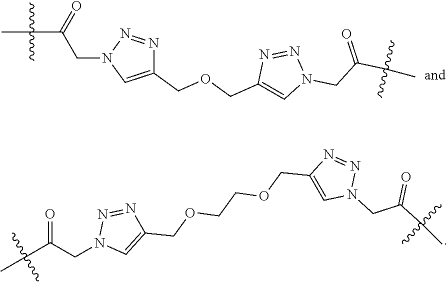 [SMILES: *C(=O)Cn1cc(COCc2cn(CC(*)=O)nn2)nn1 ; *C(=O)Cn1cc(COCCOCc2cn(CC(*)=O)nn2)nn1]

In some embodiments of Formula (VIII), L is

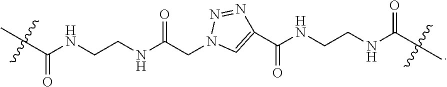 [SMILES: *C(=O)NCCNC(=O)Cn1cc(C(=O)NCCNC(*)=O)nn1]

In some embodiments of Formula (VIII), L is chosen from

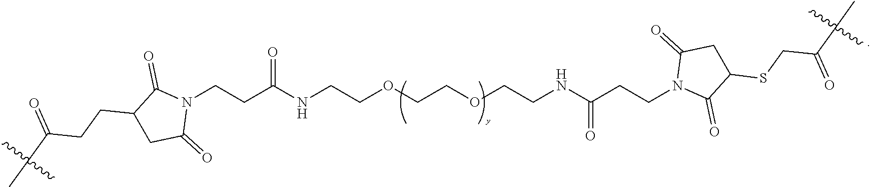

groups,
wherein y is chosen from integers ranging from 0 to 250.

In some embodiments of Formula (VIII), L is

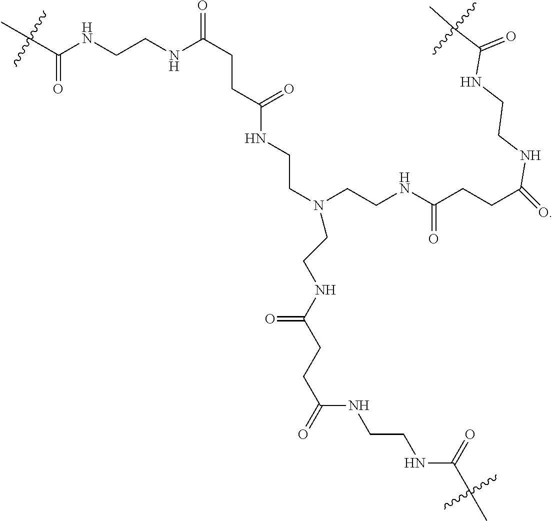 [SMILES: *C(=O)NCCNC(=O)CCC(=O)NCCN(CCNC(=O)CCC(=O)NCCNC(*)=O)CCNC(=O)CCC(=O)NCCNC(*)=O]

In some embodiments of Formula (VIII), L is

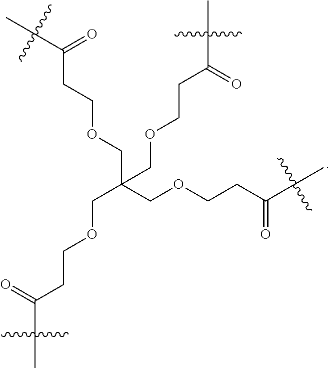 [SMILES: *C(=O)CCOCC(COCCC(*)=O)(COCCC(*)=O)COCCC(*)=O]

In some embodiments of Formula (VIII), L is

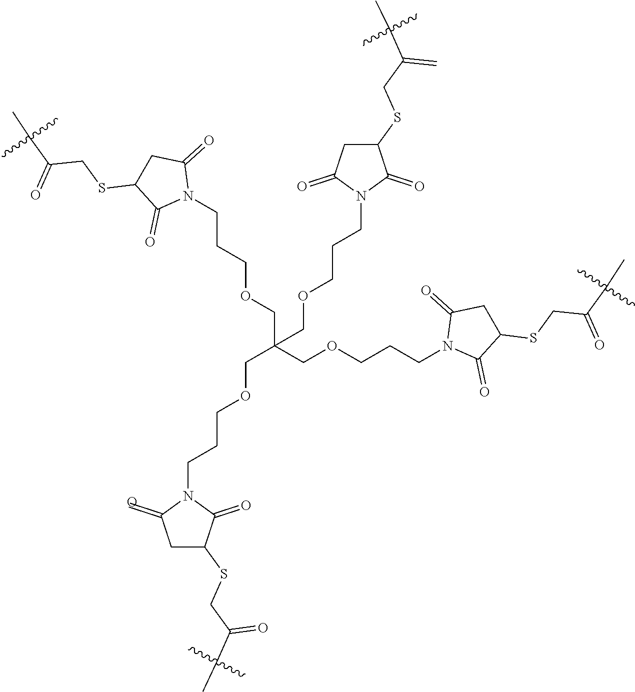

In some embodiments of Formula (VIII), L is chosen from

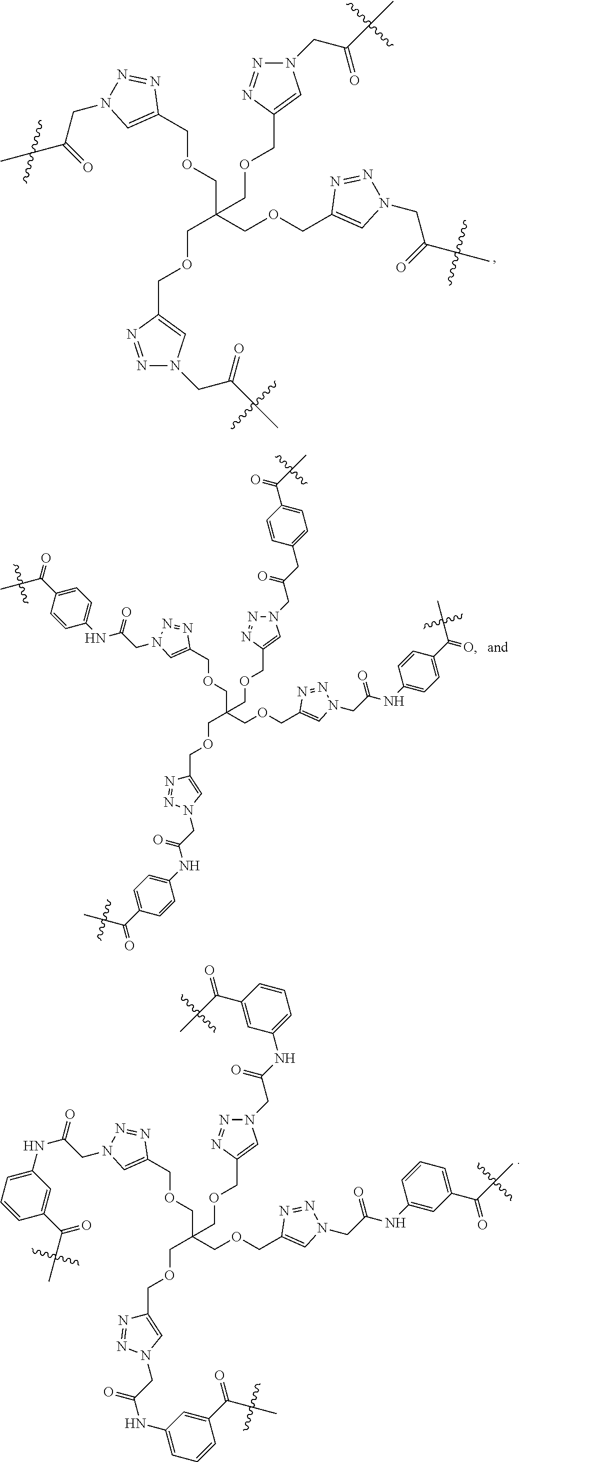 [SMILES: *C(=O)Cn1cc(COCC(COCc2cn(CC(*)=O)nn2)(COCc2cn(CC(*)=O)nn2)COCc2cn(CC(*)=O)nn2)nn1 ; "," ; *C(=O)c1ccc(CC(=O)Cn2cc(COCC(COCc3cn(CC(=O)Nc4ccc(C(*)=O)cc4)nn3)(COCc3cn(CC(=O)Nc4ccc(C(*)=O)cc4)nn3)COCc3cn(CC(=O)Nc4ccc(C(*)=O)cc4)nn3)nn2)cc1 ; "and" ; *C(=O)c1cccc(NC(=O)Cn2cc(COCC(COCc3cn(CC(=O)Nc4cccc(C(*)=O)c4)nn3)(COCc3cn(CC(=O)Nc4cccc(C(*)=O)c4)nn3)COCc3cn(CC(=O)Nc4cccc(C(*)=O)c4)nn3)nn2)c1 ; "."]

In some embodiments of Formula (VIII), L is

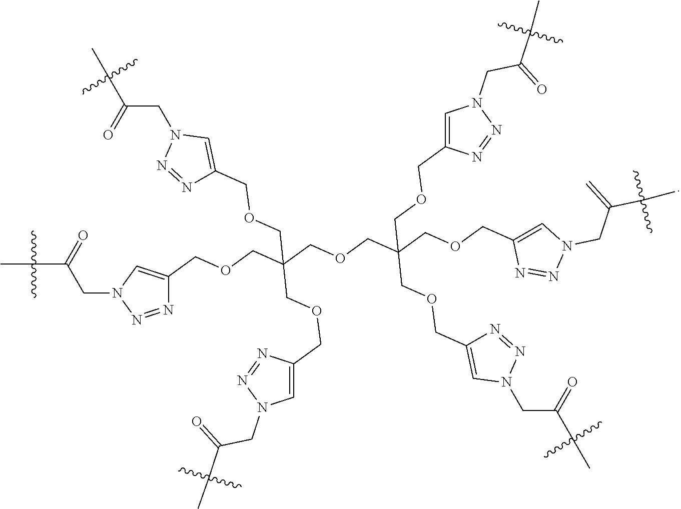 [SMILES: *C(=C)Cn1cc(COCC(COCc2cn(CC(*)=O)nn2)(COCc2cn(CC(*)=O)nn2)COCC(COCc2cn(CC(*)=O)nn2)(COCc2cn(CC(*)=O)nn2)COCc2cn(CC(*)=O)nn2)nn1]

In some embodiments of Formula (VIII), L is chosen from

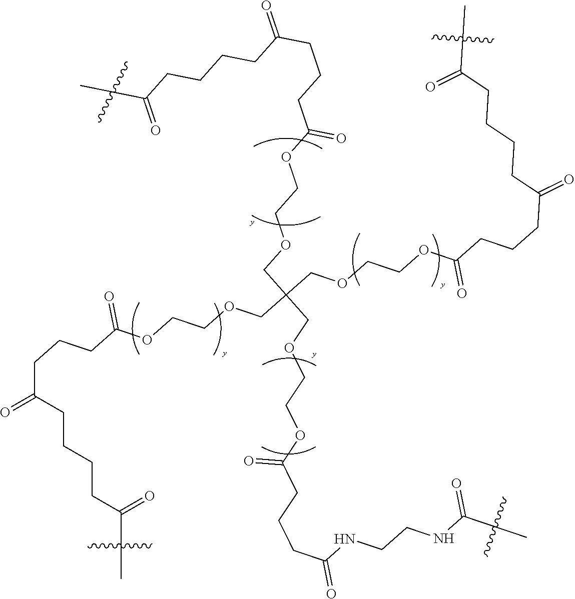

groups,
wherein each y, which may be identical or different, is independently chosen from integers ranging from 0 to 250.

In some embodiments of Formula (VIII), L is chosen from

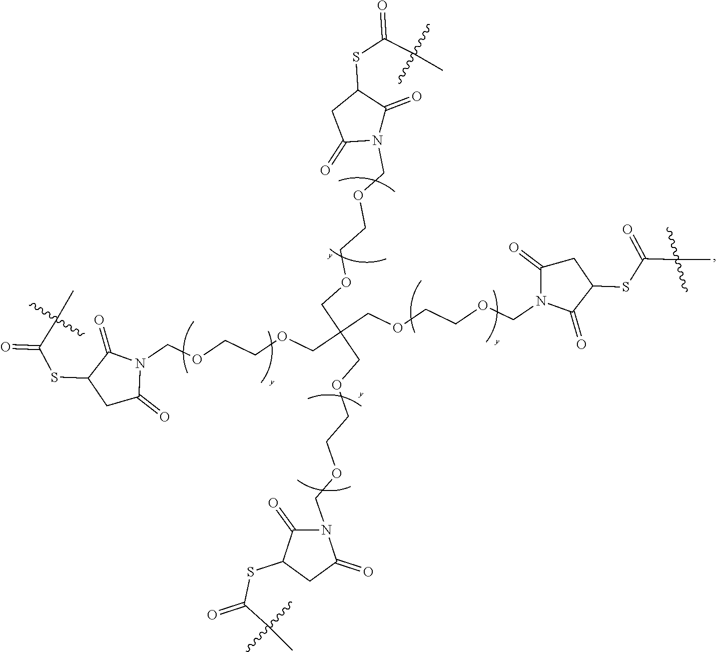

wherein each y, which may be identical or different, is independently chosen from integers ranging from 0 to 250.

In some embodiments of Formula (VIII), L is chosen from

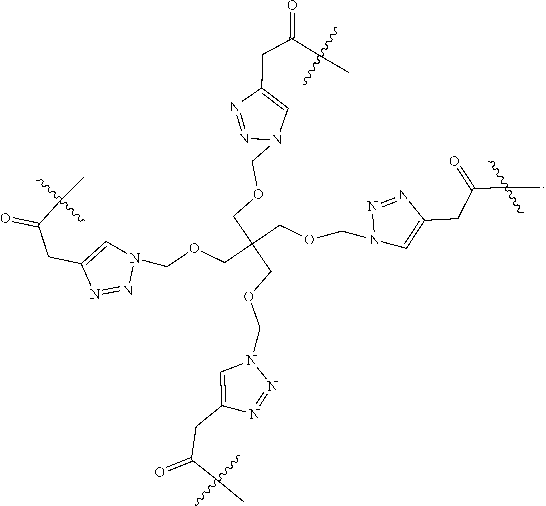 [SMILES: *C(=O)Cc1cn(COCC(COCn2cc(CC(*)=O)nn2)(COCn2cc(CC(*)=O)nn2)COCn2cc(CC(*)=O)nn2)nn1]

In some embodiments, at least one compound is chosen from compounds of Formula (VIII), wherein each R¹ is identical, each R² is identical, each R³ is identical, each R⁴ is identical, each R⁵ is identical, and each X is identical. In some embodiments, at least one compound is chosen from compounds of Formula (VIII), wherein said compound is symmetrical.

Figures and examples illustrating the synthesis of compounds of Formula (VIII) are shown in PCT International Application Publication No. WO 2020/219417, which is incorporated by reference herein.

Also provided are pharmaceutical compositions comprising at least one inhibitor chosen from compounds of Formula (I), (Ia), (II), (IIa), (III), (IIIa), (IV), (V), (IVa/Va), (IVb/Vb), (VI), (VII), and (VIII). These compounds and compositions may be used in the methods described herein. In some embodiments, provided are pharmaceutical compositions comprising at least one inhibitor chosen from Compound A, Compound B, Compound C, Compound D, and Compound E. These compounds and compositions may be used in the methods described herein.

Also provided are pharmaceutical compositions comprising at least one pharmaceutically acceptable excipient and at least one inhibitor chosen from compounds of Formula (I), (Ia), (II), (IIa), (III), (IIIa), (IV), (V), (IVa/Va), (IVb/Vb), (VI), (VII), and (VIII) and pharmaceutically acceptable salts of any of the foregoing. In some embodiments, provided are pharmaceutical compositions comprising at least one pharmaceutically acceptable excipient and at least one inhibitor chosen from Compound A, Compound B, Compound C, Compound D, and Compound E, and pharmaceutically acceptable salts of any of the foregoing. These compounds and compositions may be used in the methods described herein.

In some embodiments, the at least one inhibitor is chosen from compounds of Formula (I), (Ia), (II), (IIa), (III), (IIIa), (IV), (V), (IVa/Va), (IVb/Vb), (VI), (VII), and (VIII) and pharmaceutically acceptable salts of any of the foregoing. In some embodiments, the at least one inhibitor is chosen from compounds of Formula (I), (Ia), (II), (IIa), (III), (IIIa), (IV), (V), (IVa/Va), (IVb/Vb), (VI), (VII), and (VIII).

In some embodiments, the at least one inhibitor is Compound A. In some embodiments, the at least one inhibitor is Compound B. In some embodiments, the at least one inhibitor is Compound C. In some embodiments, the at least one inhibitor is Compound D. In some embodiments, the at least one inhibitor is Compound E.

### EXAMPLES

The following examples are intended to be illustrative and are not meant in any way to limit the scope of the disclosure.

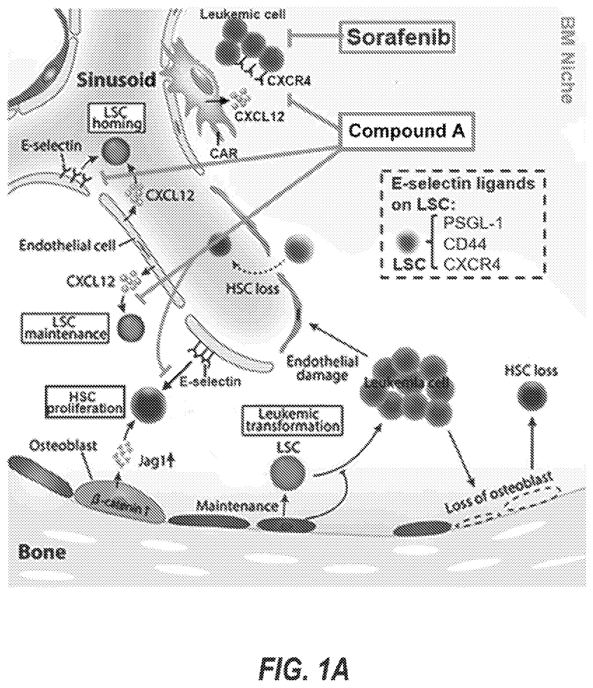

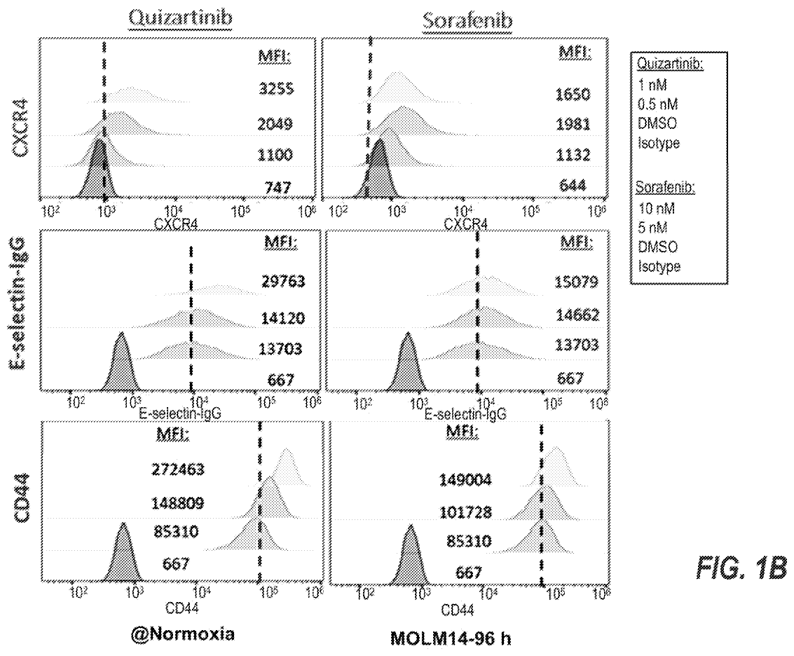

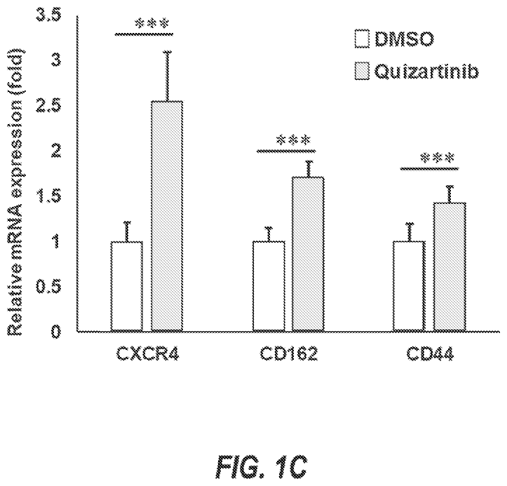

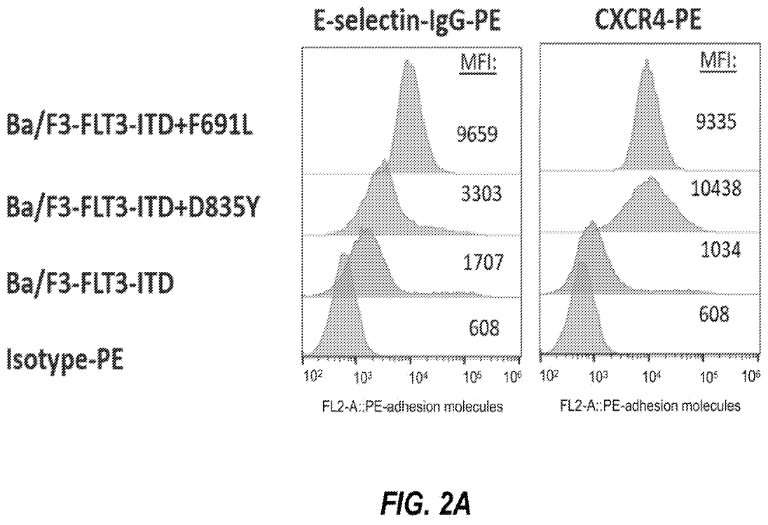

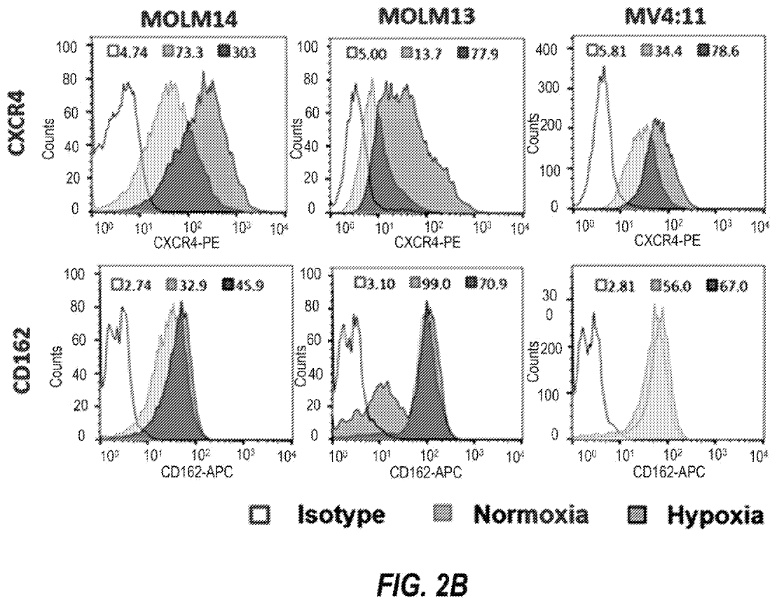

Expression levels of cell surface E-selectin ligand and CXCR4 were assessed by flow cytometry after staining with E-selectin-IgG chimera and CXCR4 in FLT3-mutated leukemia cells (FIGS. 1B-1C, 2A-2B). It was found that FLT3-mutated leukemia cells express high levels of E-selectin ligands and CXCR4, up to ˜5.6 and 10-fold, respectively.

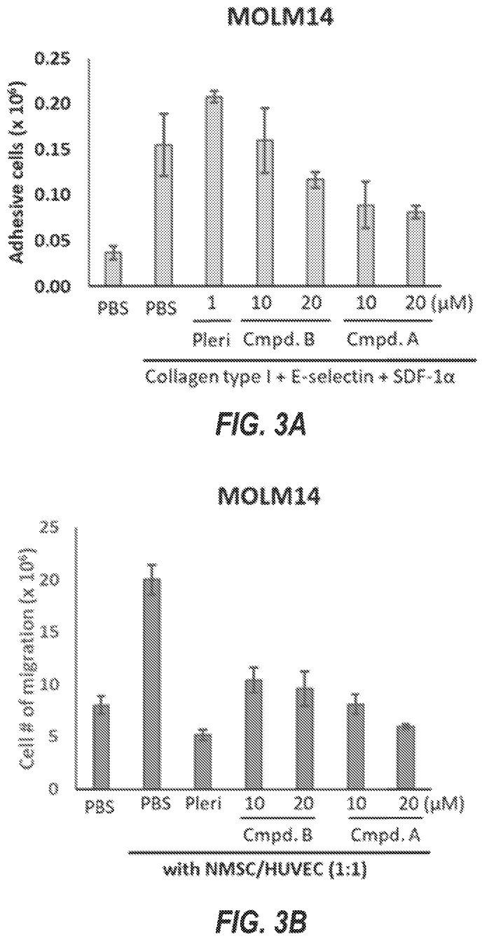

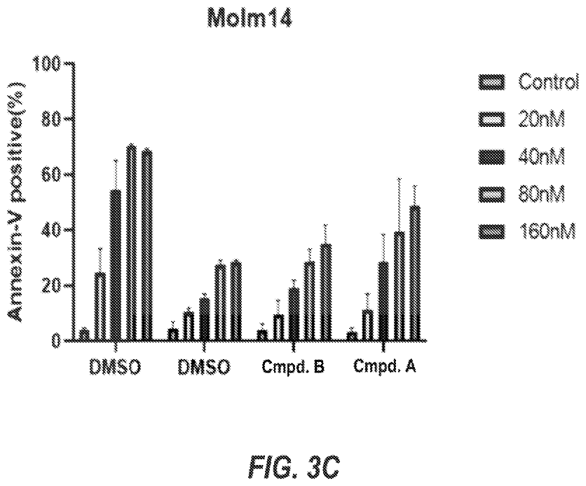

After pretreatment with E-selectin inhibitor Compound B, CXCR4 inhibitor plerixafor, or dual E-selectin/CXCR4 inhibitor Compound A for 1 hour, MOLM14 cells were seeded on pre-coated chemokine layer for 20 h. The attached cells were quantitated by Trypan blue dye exclusion. (FIG. 3A). For assessment of cell homing, cells were seeded in inserts for 16 h. The cells that migrated were determined by counting hCD45 positive cells in the outer chambers by flow cytometry. (FIG. 3B).

MOLM14 cells were treated with sorafenib for 48 h after pre-treatment with Compound B or Compound A for 2 h. Apoptosis induction was determined with flow cytometry by measuring annexin V positivity after gating on the CD45+ population. (FIG. 3C).

Blockade of E-selectin/CXCR4 with Compound A reduced leukemia cell adhesion and migration to BM niche components, and enhanced sorafenib-induced pro-apoptotic effects in vitro.

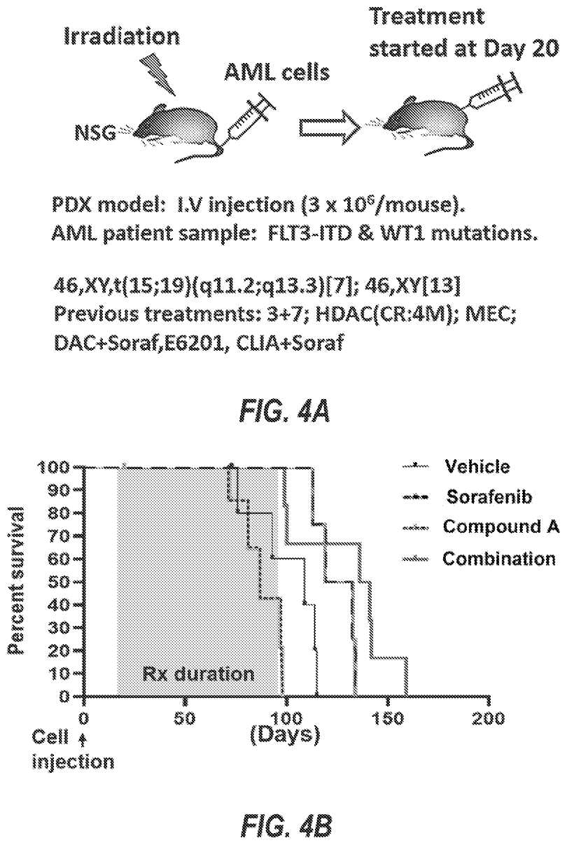

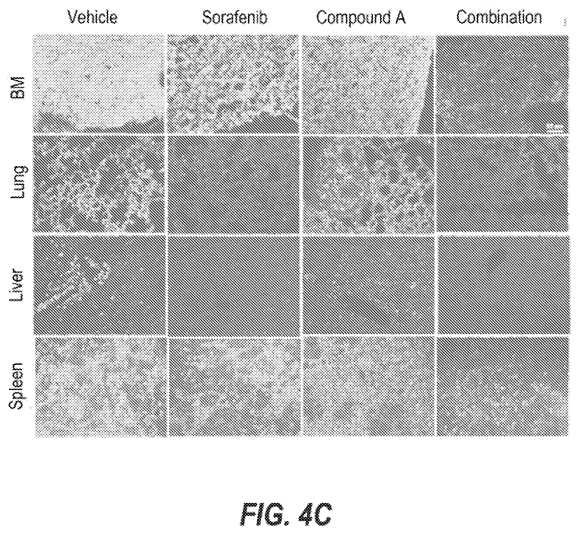

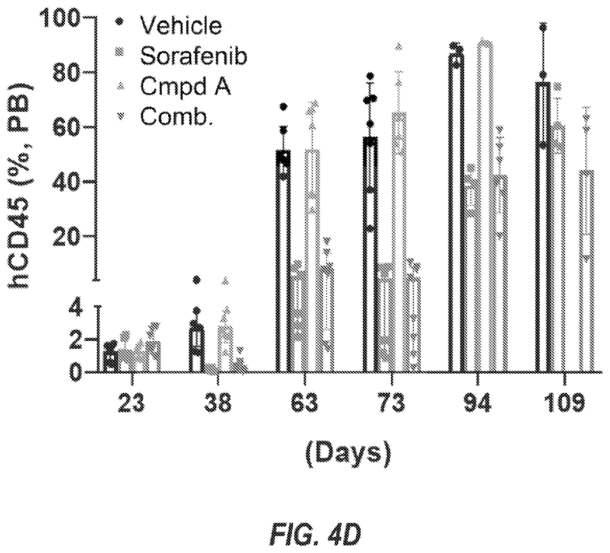

Cells from a FLT3-ITD-mutated AML patient sample were injected into irradiated NSG mice. (FIG. 4A). The patient from whom these cells were derived had been treated and relapsed after a 6 chemotherapy regimen, including sorafenib/DAC and sorafenib/CLIA. Mouse survival was estimated by the Kaplan-Meier method with log-rank statistics. (FIG. 4B). The mice started treatment with sorafenib (8 mg/kg) and/or Compound A (40 mg/kg) when leukemia cells reached 1% engraftment in PB. Engraftment was analyzed using flow cytometry measuring hCD45⁺/mCD45⁻ cells. (FIGS. 4C-4D). Co-targeting E-selectin/CXCR4 with Compound A and FLT3 with sorafenib markedly decreased leukemia cell engraftment in peripheral blood, reduced leukemia cell infiltration in the bone marrow, liver, lung, and spleen, and extended survival in an AML PDX mouse model harboring FLT3-ITD mutations.

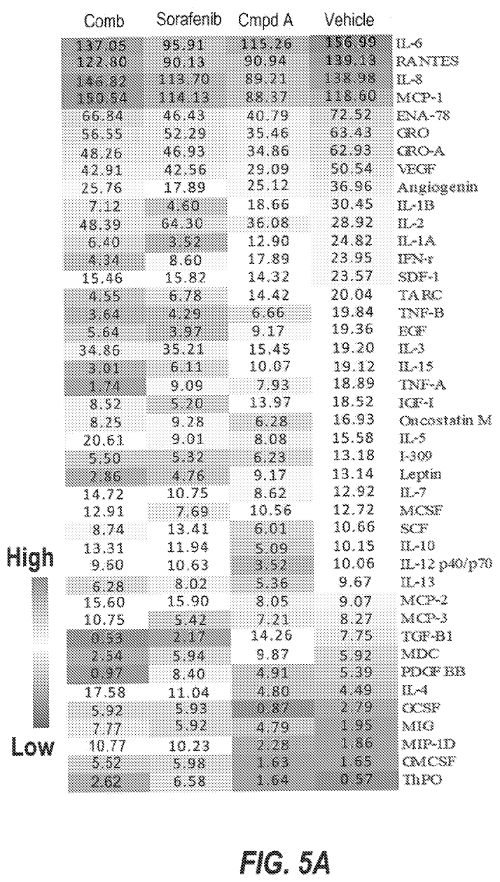

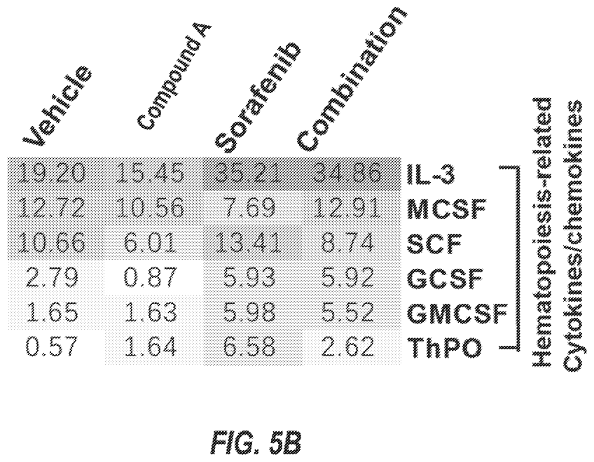

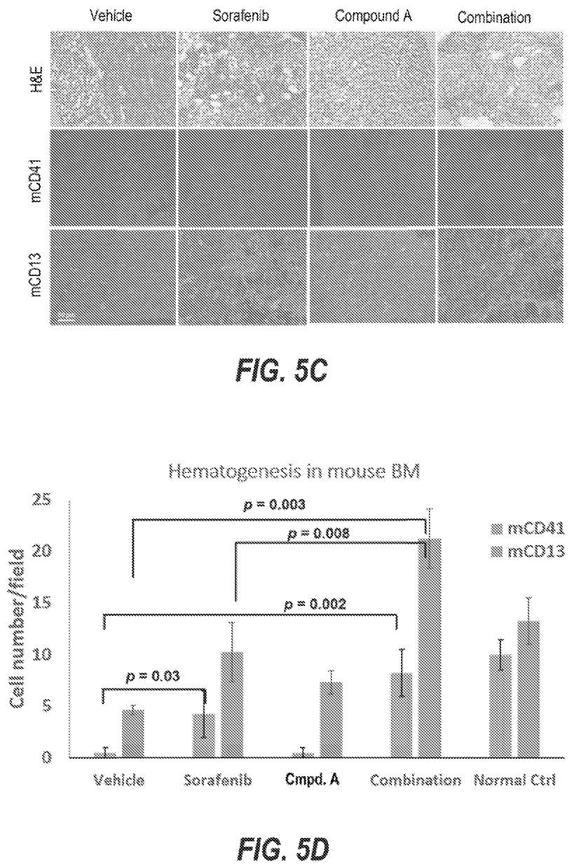

Cytokine/chemokine levels were determined with a Human Cytokine Antibody Array (Abcam, Cambridge, UK) by treating MOLM14 leukemia cells in a BM-mimetic co-culture system (MOLM14+HS-27A/HEBC-5i in hypoxia) in the presence of Compound A and/or sorafenib for 24 h, and measuring a 42-cytokine panel in the culture media (FIGS. 5A-5B).

Mouse organs were dissected at day 74 after leukemia cell injection. Tissues were prepared for routine H&E analysis and immunofluorescence (IF) staining with anti-mouse CD41, CD13 and anti-human CD45 antibodies. (FIG. 5C). Cell counts were determined by microscopy with 20× magnification. Error bars present the standard deviation for the mean counts from four random microscopy fields. (FIG. 5D). Combination therapy with Compound A and sorafenib enhanced normal hematopoiesis in mouse bone marrow by upregulating hematopoiesis-related cytokines/chemokines and increasing the numbers of megakaryocytes and myelocytes.

In another example, dual E-selectin/CXCR4 blockage was further evaluated in the context of FLT3 inhibition by sorafenib in vivo. Expression levels of E-selectin ligands and CXCR4 in FLT3 inhibitor-sensitive Ba/F3-FLT3-ITD cells and their inhibitor-resistant counterparts Ba/F3-FLT3-ITD+D835Y and Ba/F3-FLT3-ITD+F691L were compared. Resistant cells expressed 1.7 to ˜5.6-fold higher levels of total E-selectin ligand detected by a soluble E-selectin reagent, and 10-fold higher levels of CXCR4. In addition, BM-mimetic hypoxia culture profoundly upregulated the cell surface expression of PSLG-1 (P-selectin glycoprotein ligand-1, a high-affinity ligand for E-selectin) and CXCR4 on leukemia cells.

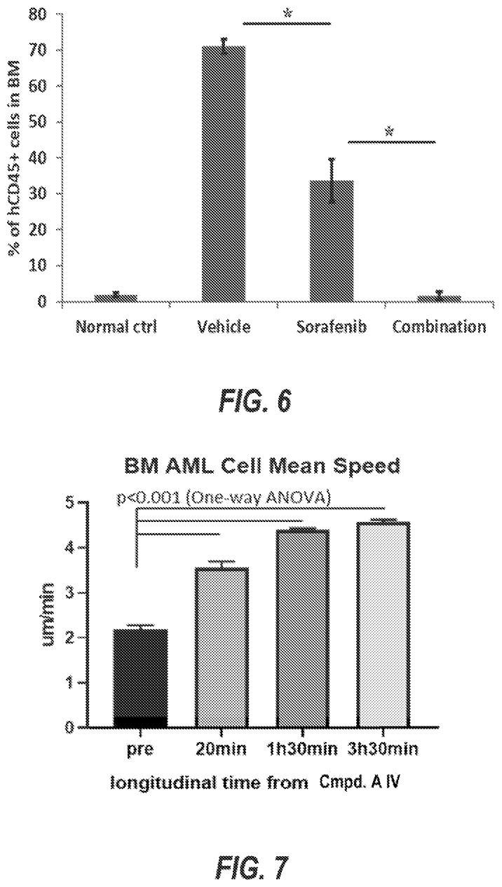

Anti-leukemia effects of co-targeting E-selectin/CXCR4 and FLT3 with Compound A and sorafenib were evaluated in a patient-derived AML xenograft (PDX) model harboring FLT3-ITD and WT1 mutations. Addition of Compound A to sorafenib greatly reduced leukemia cellularity compared to sorafenib alone, and as much as by 92%, 82%, 69% and 45% in, respectively, liver, lung, spleen and BM (FIG. 6) as compared with vehicle-treated mice (p<0.05). As expected, the number of circulating leukemia cells transiently increased. The Compound A/sorafenib combination improved mouse survival (median survival 138.5 versus 109, 87 and 126 days for the Compound A/sorafenib versus vehicle, Compound A, and sorafenib, respectively, p<0.001).

In another example, using intravital 2-photon microscopy, AML cell behavior was observed in calvarial BM with response to acute Compound A bolus infusion. Remarkably, AML cell mobility begun to increase in the BM microenvironment as soon as 20 min after treatment (FIG. 7), followed by intravasation and cellular outflow through the BM capillary vasculature over the next 2-4 hours.

Moreover, although BM homing signals are thought to be shared between leukemia and HSC, the combination therapy improved hematopoiesis parameters compared to sorafenib alone. In particular, this important effect was associated with increased numbers of megakaryocytes (2.1-fold), myelocytes (2.1-fold), and erythrocytes (7.1-fold) in BM (p<0.01). The underlying mechanism(s) of hematopoiesis protection by Compound A are under investigation.

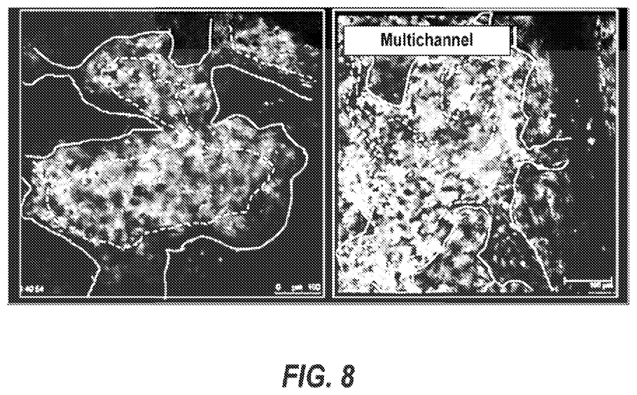

In another example, intravital 2-photon microscopy was used to study the behavioral response of AML cells to the dual CXCR4 and E-selectin inhibition by Compound A in vivo. An mTurquoise2 fluorescence-tagged transplantable mouse AML model (characterized by MLL, ENL-FLT3, ITD, p53−/−) was used. To delineate the bone and vascular niches, the syngeneic immune-competent recipient mice harbored cell lineage fluorescence reporter genes such as Col2.3-GFP, hCD2-DsRed and CD11c-EYFP with the bone collagen and blood highlighted by, respectively, second harmonic generation (SHG) and fluorescent dextran. Intravenously infused AML cells (5×10E4) homed to BM where they gradually displaced most endogenous cells. In this experimental system, either Compound A or vehicle was infused into the tail vein while recording AML cell motilities in calvarial BM stroma in 3-D over 4 hours (FIG. 8).

In untreated or control vehicle treated mice, AML cells were slowly motile, migrating with the average velocity of ˜2 μm/min. Cellular trajectories were random within the average 400 μm confinement radius corresponding to the size of BM cavities. Most AML cells localized in the vascular niche, defined as within a 50 μm distance to the nearest blood vessel. A minority of AML cells were also present in the proximity to osteoblasts and the bone (i.e., in the bone niche).

In contrast, Compound A infusion resulted in a 66% increase of the average speed of cellular motility of AML cells within 20 min and up to a 100% increase within 3.5 hours. The motility pattern remained largely random within the bone cavity confinement volume and was concentrated in the vicinity of vasculature. Multiple instances of AML cell intravasation into the capillary lumens was observed followed by several minute-long vessel wall attachment and sudden outflow. These in vivo cellular dynamics resulted in a substantial, yet structurally biased decrease of BM AML cellularity whereby the vascular niche was emptied preferentially whilst the bone niche remained populated by AML cells.

In no case was a complete BM depletion of AML cells observed. Interestingly, a single, CXCR4-only inhibitor caused a protracted (24-48 h) depletion of AML cells in BM without significant cellular motility enhancement.

These observations revealed an unexpected mechanism of action by a dual E-selectin/CXCR4 inhibitor (Compound A) involving cellular migratory motility enhancement in BM stroma prior to AML cell intravasation. Besides the designed blocking of E-selectin and CXCR4 molecules from binding their corresponding ligands, the dual moiety Compound A agent may have a capacity to generate motility-enhancing signals in AML cells that single inhibitors do not seem to trigger. Among several mechanisms possible, this action could involve E-selectin and CXCR4 crosslinking by a dual moiety molecular structure or binding avidity enhancement, requiring further investigation.

In addition, these observations highlight a likely mechanism of AML resistance to therapeutic “mobilization,” one that involves cancer cell persistence in the bone/osteoblastic niches.

Collectively, these results indicate that co-targeting E-selectin, CXCR4, and FLT3 may reduce leukemia burden and may protect normal hematopoiesis. Co-inhibition of E-selectin/CXCR4 enhances the anti-leukemia efficacy of FLT3 inhibition and preserves hematopoiesis in the BM in a PDX model of AML. For example, leukemia cells with FLT3-ITD plus TKD mutations express high levels of E-selectin ligands and CXCR4, with approximately 5.6- and 10-fold increases compared to their parental FLT3-ITD mutated cells, respectively. Additionally, blockade of E-selectin/CXCR4 with Compound A reduces leukemia cell adhesion, migration, and homing to components of the BM niche in vitro and in vivo while increasing cell motility in the vascular niche in vivo. Combination therapy with Compound A and sorafenib enhanced normal hematopoiesis in mouse BM by upregulating hematopoiesis-related cytokines and chemokines, and increased the numbers of megakaryocytes (16.5 fold) and myelocytes (4.5 fold) compared to controls. Promisingly, co-targeting E-selectin/CXCR4 with Compound A and FLT3 with sorafenib markedly decreased leukemia burden in the blood, reduced leukemia cell infiltration in the BM, liver, lung, and spleen, and extended overall survival in a FLT3-ITD-mutated AML PDX mouse model, which was resistant to 6 different treatment regimens, including 2 comprising sorafenib.

Those of ordinary skill in the art will recognize or be able to ascertain using no more than routine experimentation, many equivalents to the specific embodiments of the disclosure described herein. Such equivalents are intended to be encompassed by the following claims.

### REFERENCES

The following references are hereby incorporated by reference in their respective entireties.

Nakao M, Yokota S, Iwai T, et al. Internal tandem duplication of the flt3 gene found in acute myeloid leukemia. Leukemia. 1996 December; 10(12):1911-1918.

Kottaridis P. D. et al., Prognostic Implications of the Presence of FLT3 Mutations in Patients with Acute Myeloid Leukemia, Leukemia & Lymphoma, 2003; 44.6, 905-913.

Thiede C., et al., Analysis of FLT3-activating mutations in 979 patients with acute myelogenous leukemia: association with FAB subtypes and identification of subgroups with poor prognosis: Presented in part at the 42nd Annual Meeting of the American Society of Hematology, Dec. 1-5, 2000, San Francisco, Calif. (abstract 2334). Blood 2002; 99(12): 4326-4335.

Zhang W, K. M, Shi Y X, McQueen T, Harris D, Ling X, et al. Mutant FLT3: a direct target of sorafenib in acute myelogenous leukemia. J Natl Cancer Inst. 2008; 100:184-198.

Chien S, Haq S U, Pawlus M, et al. Adhesion of acute myeloid leukemia blasts to E-selectin in the vascular niche enhances their survival by mechanisms such as Wnt activation. Blood. 2013; 122:61.

Horacek, J. M., et al. Multi-analytical evaluation of serum levels of cytokines and adhesion molecules in patients treated for acute myeloid leukemia using biochip array technology. Biomedical Papers 2013; 157(4):277-279.

Peled A, Tavor S. Role of CXCR4 in the pathogenesis of acute myeloid leukemia. Theranostics. 2013; 3(1):34-39.

Zhang W et al., Dual E-Selectin/CXCR4 Antagonist GMI-1359 Exerts Efficient Anti-Leukemia Effects in a FLT3 ITD Mutated Acute Myeloid Leukemia Patient-Derived Xenograft Murine Model. Blood 2016; 128(22): 3519.

Zhang W et al., The Dual E-Selectin/CXCR4 Inhibitor, GMI-1359, Enhances Efficacy of Anti-Leukemia Chemotherapy in FLT3-ITD Mutated Acute Myeloid Leukemia. Blood 2015; 126 (23): 3790.

Kupsa T. et al., Serum levels of soluble adhesion molecules in newly diagnosed acute myeloid leukemia and in complete remission suggest endothelial cell activation by myeloblasts, Biomed Pap Med Fac Univ Palacky Olomouc Czech Repub. 2016; 160:94-99.

Roboz et al., Current treatment of acute myeloid leukemia, Curr. Opin. Oncol. 2012; 24:711-719.

Burnett et al., Attempts to optimize induction and consolidation treatment in acute myeloid leukemia: results of the MRC AML 12 trial, J. Clin. Oncol. 2010; 28:586-595.

Fernandez et al., Anthracycline Dose Intensification in Acute Myeloid Leukemia, N. Engl. J. Med. 2009; 361:1249-1259.

Mandelli et al., Daunorubicin Versus Mitoxantrone Versus Idarubicin As Induction and Consolidation Chemotherapy for Adults With Acute Myeloid Leukemia: The EORTC and GIMEMA Groups Study AML-10, J. Clin. Oncol. 2009; 27:5397-5403.

Ravandi et al., Eradication of Leukemia Stem Cells as a New Goal of Therapy in Leukemia, Clin. Can. Res. 2006; 12(2):340-344.

Dean et al., Tumour stem cells and drug resistance, Nat. Rev. Cancer 2005; 5(4):275-294.

Guan et al., Detection, isolation, and stimulation of quiescent primitive leukemic progenitor cells from patients with acute myeloid leukemia (AML), Blood 2003; 101(8):3142.

Konopleva et al., The anti-apoptotic genes Bcl-XL and Bcl-2 are over-expressed and contribute to chemoresistance of non-proliferating leukaemic CD34+ cells, Br. J. Haematol. 2002; 118(2):521-534.

Falini et al., New Classification of Acute Myeloid Leukemia and Precursor-related Neoplasms: Changes and Unsolved Issues, Discov. Med. 2010; 10(53):281-92.

Lee et al., Minimally Differentiated Acute Nonlymphocytic Leukemia: A Distinct Entity, Blood 1987; 70(5):1400-1406.

Hendrix et al., Pharmacokinetics and safety of AMD-3100, a novel antagonist of the CXCR-4 chemokine receptor, in human volunteers, Antimicrob. Agents Chemother. 2000; 44:1667-1673.

Doranz et al., Safe use of the CXCR4 inhibitor ALX40-4C in humans, AIDS Research and Human Retroviruses 2001; 17:475-486.

Arakaki et al., T134, a small molecule CXCR4 inhibitor, has no cross drug-resistance with the different class of CXCR4 antagonist AMD3100, J. Virol. 1999; 73:1719-1723.

1. A method of treating a cancer in a subject in need thereof comprising administering to the subject at least one FLT3 inhibitor and at least one inhibitor chosen from E-selectin inhibitors, CXCR4 inhibitors, and heterobifunctional inhibitors of E-selectin and CXCR4.

2. (canceled)

3. (canceled)

4. The method according to claim 1, wherein the subject is a human.

5. The method according to claim 1, wherein the subject is a cancer patient.

6. The method according to claim 1, wherein the subject is a relapsed cancer patient.

7. The method according to claim 1, wherein the subject is receiving, has received, or will receive chemotherapy and/or radiotherapy.

8. The method according to claim 1, wherein the subject is receiving, has received, or will receive MMP inhibitors, inflammatory cytokine inhibitors, mast cell inhibitors, NSAIDs, NO inhibitors, MDM2 inhibitors, or antimicrobial compounds.

9. The method according to claim 1, wherein the cancer is chosen from liquid cancers.

10. The method according to claim 1, wherein the cancer is chosen from solid cancers.

11. The method according to claim 1, wherein the cancer is chosen from colorectal cancer, liver cancer, gastric cancer, lung cancer, brain cancer, kidney cancer, bladder cancer, thyroid cancer, prostate cancer, ovarian cancer, cervical cancer, uterine cancer, endometrial cancer, breast cancer, pancreatic cancer, leukemia, lymphoma, myeloma, melanoma, kidney chromophobe carcinoma, adrenocortical carcinoma, bladder urothelial carcinoma, thymoma, testicular germ cell tumors, and head and neck squamous cell carcinoma.

12. The method according to claim 1, wherein the cancer is chosen from FLT3 mutated cancers.

13. The method according to claim 1, wherein the cancer is chosen from FLT3-ITD mutated cancers.

14. The method according to claim 1, wherein the cancer is AML.

15. The method according to claim 1, wherein the cancer is relapsed/refractory AML.

16. The method according to claim 1, wherein the cancer is FLT3-ITD mutated AML.

17. The method according to claim 1, wherein the subject possesses one or more mutational alterations of FLT3.

18. The method according to claim 17, wherein the mutational alterations are chosen from internal tandem duplications and missense mutations within the tyrosine kinase domain activation loop of FLT3.

19. The method according to claim 1, wherein the blast cells in the subject have an increased gene expression level of FUT7 relative to a control sample from a non-cancer subject, a newly diagnosed cancer subject, or a subject having the same cancer as the patient.

20. The method according to claim 1, comprising administering to the subject at least one FLT3 inhibitor and at least one E-selectin inhibitor.

21. The method according to claim 1, comprising administering to the subject at least one FLT3 inhibitor and at least one CXCR4 inhibitor.

22. The method according to claim 1, comprising administering to the subject at least one FLT3 inhibitor and at least one heterobifunctional inhibitor of E-selectin and CXCR4.

23. The method according to claim 1, comprising administering to the subject at least one FLT3 inhibitor, at least one E-selectin inhibitor, and at least one CXCR4 inhibitor.

24. The method according to claim 1, wherein the at least one inhibitor is chosen from 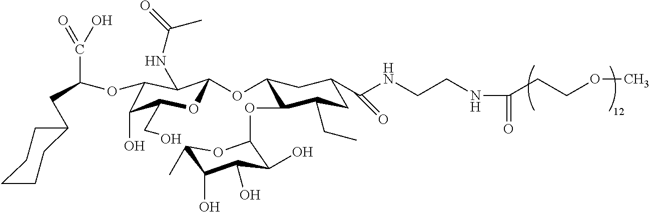
and pharmaceutically acceptable salts thereof.

25. The method according to claim 1, wherein the at least one inhibitor is

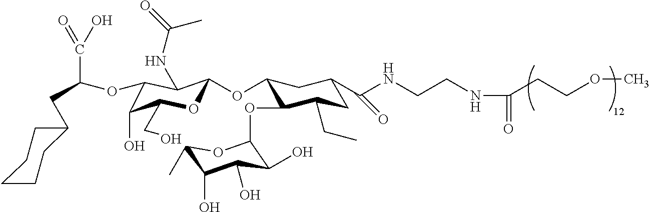

26. The method according to claim 1, wherein the at least one inhibitor is chosen from 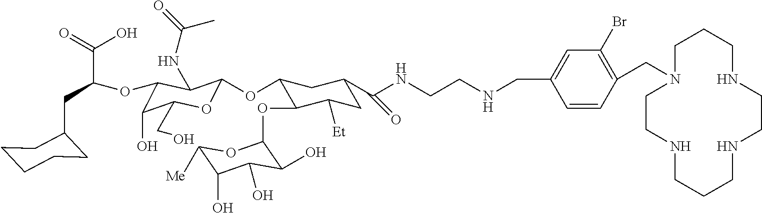 [SMILES: CCC1CC(C(=O)NCCNCc2ccc(CN3CCCNCCNCCCNCC3)c(Br)c2)CC(OC2OC(CO)C(O)C(O[C@@H](CC3CCCCC3)C(=O)O)C2NC(C)=O)C1OC1OC(C)C(O)C(O)C1O]
and pharmaceutically acceptable salts thereof.

27. The method according to claim 1, wherein the at least one inhibitor is

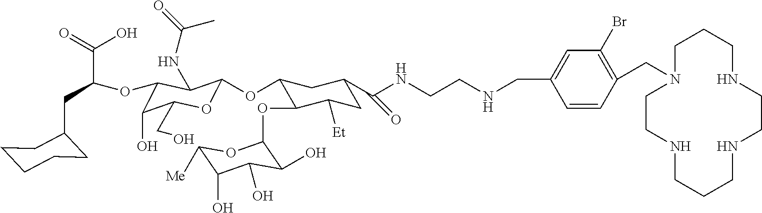 [SMILES: CCC1CC(C(=O)NCCNCc2ccc(CN3CCCNCCNCCCNCC3)c(Br)c2)CC(OC2OC(CO)C(O)C(O[C@@H](CC3CCCCC3)C(=O)O)C2NC(C)=O)C1OC1OC(C)C(O)C(O)C1O]

28. The method according to claim 24, wherein the method comprises administering a dose in the range of 5 mg/kg to 100 mg/kg of the at least one inhibitor.

29. The method according to claim 24, wherein the method comprises administering a fixed dose of 20 mg to 4000 mg per day of the at least one inhibitor.

30. The method according to claim 28, wherein the method comprises administering a dose in the range of 0.5 mg/kg to 100 mg/kg of the at least one FLT3 inhibitor.

31. The method according to claim 29, wherein the method comprises administering a fixed dose of 20 mg to 4000 mg per day of the at least FLT3 inhibitor.

32. The method according to claim 1, wherein the at least one FLT3 inhibitor is chosen from first generation multi-kinase inhibitors and next generation inhibitors.

33. The method according to claim 1, wherein the at least one FLT3 inhibitor is chosen from sorafenib, lestaurtinib, midostaurin, quizartinib, crenolanib, gilteritinib, and pharmaceutically acceptable salts of any of the foregoing.

34. The method according to claim 1, wherein the at least one FLT3 inhibitor is sorafenib.

35. The method according to claim 1, wherein the at least one FLT3 inhibitor is quizartinib.

36. The method according to claim 27, wherein the method comprises administering a dose in the range of 5 mg/kg to 100 mg/kg of the at least one inhibitor.

37. The method according to claim 27, wherein the method comprises administering a fixed dose of 20 mg to 4000 mg per day of the at least one inhibitor.

38. The method according to claim 36, wherein the method comprises administering a dose in the range of 0.5 mg/kg to 100 mg/kg of the at least one FLT3 inhibitor.

39. The method according to claim 37, wherein the method comprises administering a fixed dose of 20 mg to 4000 mg per day of the at least FLT3 inhibitor.
